(19) United States
(12) Patent Application Publication
(10) Pub. No.: US 2011/0189676 A1
(43) Pub. Date: Aug. 4, 2011

(54) COMPOSITIONS FOR USE IN IDENTIFICATION OF FUNGI

(76) Inventors: Thomas A. Hall, Oceanside, CA (US); Mark Frinder, San Clemente, CA (US); Raymond Ranken, Encinitas, CA (US); Miriam Reyes, Irvine, CA (US); Sherilynn Manalili Wheeler, Encinitas, CA (US); Lawrence B. Blyn, Mission Viejo, CA (US); Rangarajan Sampath, San Diego, CA (US); David J. Ecker, Encinitas, CA (US)

(21) Appl. No.: 13/122,367

(22) PCT Filed: Sep. 30, 2009
(86) PCT No.: PCT/US09/59010

§ 371 Date: Apr. 1, 2011

Related U.S. Application Data
(60) Provisional application No. 61/102,628, filed on Oct. 3, 2008.

(51) Int. Cl. C12Q 1/68; C12M 1/36

(52) U.S. Cl. 435/6.11; 435/6.15; 435/286.1

(57) ABSTRACT

The present invention relates generally to identification of fungi pathogens, and provides methods, compositions, systems and kits useful for this purpose when combined, for example, with molecular mass or base composition analysis.

### CROSS REFERENCE TO RELATED APPLICATIONS

The present Application claims priority to U.S. Provisional Application No. 61/102,628, filed Oct. 3, 2008, which is incorporated by reference in its entirety.

### FIELD OF THE INVENTION

The present invention relates generally to the detection and identification of fungi, and provides methods, compositions, systems and kits useful for this purpose when combined, for example, with molecular mass or base composition analysis.

### BACKGROUND OF THE INVENTION

Fungal diseases are called mycoses and those affecting humans can be divided into four groups based on the level of penetration into the body tissues: Superficial mycoses are caused by fungi that grow only on the surface of the skin or hair. Cutaneous mycoses or dermatomycoses include such infections as athlete's foot and ringworm, in which growth occurs only in the superficial layers of skin, nails, or hair. Subcutaneous mycoses penetrate below the skin to involve the subcutaneous, connective, and bone tissue. Systemic or deep mycoses are able to infect internal organs and become widely disseminated throughout the body. This type is often fatal.

The most common type of subcutaneous mycosis seen worldwide is sporotrichosis, which occurs most often in gardeners and farmers who come in direct contact with soil. This is a chronic infection caused by the fungus Sporothrix schenckii, occurring in three forms. The ‘cutaneous lymphatic form’ is characterized by a single pustule or nodule that forms at the site of invasion. This is followed by lymphatic spread and the development of numerous subcutaneous lesions. This ‘disseminated form’ is marked by multiple, painless cutaneous or subcutaneous nodules, which can form into ulcers or abscesses involving the muscles, joints, bones, eyes, gastrointestinal system, mucous membranes, and nervous system. The ‘pulmonary form’ results from the inhalation of spores, but produces much the same forms of the disease.

Other forms of subcutaneous mycoses occur mostly in the tropics and subtropics and are caused by several fungal species. These conditions are called chromomycosis (producing wartlike nodules that can ulcerate) and maduromycosis (or mycetoma—a chronic slowly progressive destructive infection involving several layers of skin, producing abscessing granulomas). Treatment is difficult and often requires surgical removal of the offending tissues.

Dermatomycoses is a superficial fungal infection that penetrates only the epidermis, hair, or nails. About thirty different species of the genera Epidermophyton, Microsporum, and Trichophyton (collectively known as dermatophytes) cause infections commonly known as athlete's foot, jock itch, and ringworm.

Systemic mycoses occur in two basic forms—respiratory and disseminated tissue. If left untreated, the disseminated form is usually fatal. Surgical removal of large pulmonary lesions have been useful in some cases. Other systemic mycoses include: occidioidomycosis (valley fever), caused by Coccidioides immitis; histoplasmosis, caused by the fungus Histoplasma capsulatum; blastomycosis, caused by the fungus Blastomycoses dermatitidis; and cryptococcosis, caused by the yeast Cryptococcus neoformans.

The fungus, Coccidioides immitis is an infectious, but not contagious, disease contracted by inhaling spores produced in soil. The fungi grow so rapidly that death can result very quickly. Once inhaled, the spores often migrate to one area and lay dormant in a pocket of tissue, looking like a hanging beehive. Amphotericin B is an effective drug, but it has significant side effects, killing healthy human cells as well as the invasive agent. This may leave the kidneys and bone marrow especially sensitive, and often results in kidney damage and anemia.

Mucormycosis refers to the fairly rare diseases produced by a variety of common fungi of the order Mucorales. These are seen only in the severely immunocompromised patient. The fungi penetrate the respiratory or intestinal mucosa and can enter through breaks in the skin as well. Localized lesions may develop, followed by a spreading to the blood, which carry it to all organs. Death often results.

Fungi most commonly associated with specific immunocompromised patients include Candida species, Aspergillus species, Phycomyces species (leucopenia or bone marrow failure); Candida, Cryptococcus, Coccidioides, Histoplasma (cellular immunity or tissue transplants); Zygomyces, Rhizopus, Mucor, Absidia (diabetes); Zygomyces (steroid therapy); Candida, Cryptococcus, Histoplasma (malignancies as in leukemia and lymphoma, and Hodgkin's disease); and Candida, Cryptococcus, Histoplasma (acquired immunodeficiency syndrome).

Diagnosis of candidemia typically relies upon blood cultures, which often require several days for correct species identification. Diagnosis of invasive fungal infection (IFI) relies upon a consensus of clinical and laboratory criteria with certainty ranging from definite to probable or possible. Because definite diagnosis requires observation of the organism in tissue, a significant proportion of patients with IFI fall into the probable or possible categories.

What is needed are improved compositions, methods, systems and kits for detecting and identifying fungi at the order, genus and/or species levels from, for example, fungal and yeast culture isolates, bronchoalveolar lavage samples, transplanted tissue samples, direct tissue biopsy samples, blood and sputum samples, and in swabs (e.g., nasal, throat) or skin scrapings, and/or detection and identification of molecular markers for anti-fungal drug resistance (e.g., fluconazole, 5-Flucytosine).

### SUMMARY OF THE INVENTION

The present invention relates generally to the detection and identification of fungi, and provides methods, compositions and kits useful for this purpose when combined, for example, with molecular mass or base composition analysis. However, the compositions and methods find use in a variety of biological sample analysis techniques and are not limited to processes that employ or require molecular mass or base composition analysis. For example, primers described herein find use in a variety of research, surveillance, and diagnostic approaches that utilize one or more primers, including a variety of approaches that employ the polymerase chain reaction.

Tor further illustrate, in certain embodiments the invention provides for the rapid detection and identification of fungi. In one aspect, the present invention provides a composition comprising at least one purified oligonucleotide primer pair that comprises forward and reverse primers, wherein said primer pair comprises nucleic acid sequences that are substantially complementary to nucleic acid sequences of two or more different bioagents belonging to the fungi family/genus/species/strain, wherein the primer pair is configured to produce amplicons comprising different base compositions that correspond to the two or more different bioagents. In addition to compositions and kits that include one or more of the primer pairs described herein, the invention also provides related methods and systems.

In some embodiments, the present invention provides compositions comprising at least one purified oligonucleotide primer pair that comprises forward and reverse primers about 15 to 35 nucleobases in length, wherein the forward primer comprises at least 70% identity (e.g., 70% . . . 75% . . . 90% . . . 95% . . . 100%) with a sequence selected from SEQ ID NOs:1-70, 141, 143, 145, 147, 149 and 151 and wherein the reverse primer comprises at least 70% identity (e.g., 70% . . . 75% . . . 90% . . . 95% . . . 100%) with a sequence selected from SEQ ID NOs:71-140, 142, 144, 146, 148, 150 and 152. In certain embodiments, the primer pair is configured to hybridize with fungi. In further embodiments, the primer pair is selected from the group of primer pair sequences consisting of: SEQ ID NOs: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, and 70:140, 141:142, 143:144, 145:146, 147:148, 149:150 and 151:152. In certain embodiments, the forward and/or reverse primer has a base length selected from the group consisting of:15, 16, 17, 18, 19, 20, 21, 22, 23, 24, 25, 26, 27, 28, 29, 30, 31, 32, 33, 34, or 34 nucleotides, although both shorter and longer primers may be used.

In another aspect, the invention provides a purified oligonucleotide primer pair, comprising a forward primer and a reverse primer that each independently comprise 14 to 40 consecutive nucleobases selected from the primer pair sequences shown in Table 1, which primer pair is configured to generate an amplicon between about 50 and 150 consecutive nucleobases in length.

In another aspect, the invention provides a kit comprising at least one purified oligonucleotide primer pair that comprises forward and reverse primers that are about 20 to 35 nucleobases in length, and wherein the forward primer comprises at least 70%, at least 80%, at least 90%, at least 95%, or at least 100% sequence identity with a sequence selected from the group consisting of SEQ ID NOs: 1-70, 141, 143, 145, 147, 149 and 151 and the reverse primer comprises at least 70% sequence identity (e.g., 75%, 85%, or 95%) with a sequence selected from the group consisting of SEQ ID NOs: 71-140, 142, 144, 146, 148, 150 and 152. In some embodiments, the kit comprises a primer pair that is a broad range survey primer pair (e.g., specific for nucleic acid of a housekeeping gene found in many or all members of a category of organism, such as ribosomal RNA encoding genes in fungus). Examples of broad range survey primers include, but are not limited to: primer pair numbers: 3815 (SEQ ID NOs: 6:76) and 3816 (SEQ ID NOs: 7:77), which target DNA encoding 5.8S rRNA.

In other embodiments, the amplicons produced with the primers are 45 to 200 nucleobases in length (e.g., 45 . . . 75 . . . 125 . . . 175 . . . 200). In some embodiments, a non-templated T residue on the 5′-end of said forward and/or reverse primer is removed. In still other embodiments, the forward and/or reverse primer further comprises a non-templated T residue on the 5′-end. In additional embodiments, the forward and/or reverse primer comprises at least one molecular mass modifying tag. In some embodiments, the forward and/or reverse primer comprises at least one modified nucleobase. In further embodiments, the modified nucleobase is 5-propynyluracil or 5-propynylcytosine. In other embodiments, the modified nucleobase is a mass modified nucleobase. In still other embodiments, the mass modified nucleobase is 5-Iodo-C. In additional embodiments, the modified nucleobase is a universal nucleobase. In some embodiments, the universal nucleobase is inosine. In certain embodiments, kits comprise the compositions described herein.

In particular embodiments, the present invention provides methods of determining the presence of a fungus in at least one sample, the method comprising: (a) amplifying one or more (e.g., two or more, three or more, four or more, etc.; one to two, one to three, one to four, etc.; two, three, four, etc.) segments of at least one nucleic acid from the sample using at least one purified oligonucleotide primer pair that comprises forward and reverse primers that are about 20 to 35 nucleobases in length, and wherein the forward primer comprises at least 70% (e.g., 70% . . . 75% . . . 90% . . . 95% . . . 100%) sequence identity with a sequence selected from the group consisting of SEQ ID NOs:1-70, 141, 143, 145, 147, 149 and 151 and the reverse primer comprises at least 70% (e.g., 70% . . . 75% . . . 90% . . . 95% . . . 100%) sequence identity with a sequence selected from the group consisting of SEQ ID NOs:71-140, 142, 144, 146, 148, 150 and 152 to produce at least one amplification product; and (b) detecting the amplification product, thereby determining the presence of the fungi in the sample.

In certain embodiments, step (b) comprises determining an amount of (i.e., the quantity) the fungi in the sample. In further embodiments, step (b) comprises detecting a molecular mass of the amplification product. In other embodiments, step (b) comprises determining a base composition of the amplification product, wherein the base composition identifies the number of A residues, C residues, T residues, G residues, U residues, analogs thereof and/or mass tag residues thereof in the amplification product, whereby the base composition indicates the presence of the fungi in the sample or identifies the pathogenicity of the fungi in the sample. In particular embodiments, the methods further comprise comparing the base composition of the amplification product to calculated or measured base compositions of amplification products of one or more known fungi present in a database, for example, with the proviso that sequencing of the amplification product is not used to indicate the presence of or to identify the fungi, wherein a match between the determined base composition and the calculated or measured base composition in the database indicates the presence of or identifies the fungi. In some embodiments, the identification of fungi is at the biological kingdom level, phylum level, class level, order level, family level, genus level, species level, sub-type level (e.g., strain level), genotype level, or individual identity level.

In some embodiments, the present invention provides methods of identifying one or more fungal bioagents in a sample, the method comprising: amplifying two or more segments of a nucleic acid from the one or more fungal bioagents in the sample with two or more oligonucleotide primer pairs to obtain two or more amplification products (e.g., from a single bioagent); (b) determining two or more molecular masses and/or base compositions of the two or more amplification products; and (c) comparing the two or more molecular masses and/or the base compositions of the two or more amplification products with known molecular masses and/or known base compositions of amplification products of known fungal bioagents produced with the two or more primer pairs to identify the one or more fungal bioagents in the sample. In certain embodiments, the methods comprise identifying the one or more fungal bioagents in the sample using three, four, five, six, seven, eight or more primer pairs. In other embodiments, the one or more fungal bioagents in the sample cannot be identified using a single primer pair of the two or more primer pairs. In particular embodiments, the methods comprise obtaining the two or more molecular masses of the two or more amplification products via mass spectrometry. In certain embodiments, the methods comprise calculating the two or more base compositions from the two or more molecular masses of the two or more amplification products. In some embodiments, the fungal bioagents are selected from the group consisting of a fungal genus, a species thereof, a sub-species thereof, and combinations thereof.

In some embodiments, the present invention provides methods of identifying one or more strains of fungi in a sample, the method comprising: (a) amplifying two or more segments of a nucleic acid from the one or more fungi in the sample with first and second oligonucleotide primer pairs to obtain two or more amplification products, wherein the first primer pair is a broad range survey primer pair, and wherein the second primer pair produces an amplicon that reveals species, sub-type, strain, or genotype-specific information; (b) determining two or more molecular masses and/or base compositions of the two or more amplification products; and (c) comparing the two or more molecular masses and/or the base compositions of the two or more amplification products with known molecular masses and/or known base compositions of amplification products of known fungi produced with the first and second primer pairs to identify the fungi in the sample. In some embodiments, the second primer pair amplifies a portion of a gene including, but not limited to 25S rRNA, actin, mito-ss rRNA, EFT-1a, EFT2, GSC1, pyroA, mito-ssu rRNA, 18S (ssu) rRNA, mito-cytochrome B, chitin synthase, mito-1su rRNA, major surface glycoprotein, beta-tubulin, and topoisomerase II.

In certain embodiments, the second primer pair comprises forward and reverse primers that are about 20 to 35 nucleobases in length, and wherein the forward primer comprises at least 70% sequence identity with a sequence selected from the group consisting of SEQ ID NOs:1-70, 141, 143, 145, 147, 149 and 151 and the reverse primer comprises at least 70% sequence identity with a sequence selected from the group consisting of SEQ ID NOs:71-140, 142, 144, 146, 148, 150 and 152 to produce at least one amplification product. In further embodiments, the obtaining the two or more molecular masses of the two or more amplification products is via mass spectrometry. In some embodiments, the methods comprise calculating the two or more base compositions from the two or more molecular masses of the two or more amplification products. In further embodiments, the fungi are selected from the group consisting of: Candida spp. (e.g., Candida albicans, Candida tropicalis, Candida glabrata, Candida parapsilosis and Candida crusei), Aspergillus spp., Fusarium spp. (e.g., Fusarium solani and Fusarium oxysporum), Cryptococus spp. (e.g., Cryptococcus neoformans), Coccidiodes spp. (e.g., Coccidiodes immitis, Coccidiodes posadasii), Mucor spp., Rhizopus spp., Mucorales (e.g., Mucoraceae, Ajellomyces spp.), and Pneumocystis jirovecii.

In some embodiments, the second primer pair is selected from the group of primer pair sequences consisting of: SEQ ID NOs: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, 70:140, 141:142, 143:144, 145:146, 147:148, 149:150 and 151:152.

In other embodiments, the determining the two or more molecular masses and/or base compositions is conducted without sequencing the two or more amplification products. In certain embodiments, the fungi in the sample cannot be identified using a single primer pair of the first and second primer pairs. In other embodiments, the fungi in the sample is identified by comparing three or more molecular masses and/or base compositions of three or more amplification products with a database of known molecular masses and/or known base compositions of amplification products of known fungi produced with the first and second primer pairs, and a third primer pair.

In further embodiments, members of the first and second primer pairs hybridize to conserved regions of the nucleic acid that flank a variable region. In some embodiments, the variable region varies between at least two species of fungi. In particular embodiments, the variable region uniquely varies between at least two (e.g., 3, 4, 5, 6, 7, 8, 9, 10, . . . , 20, etc.) genera, species, sub-types, strains, or genotypes of fungi.

In some embodiments, the present invention provides systems comprising: (a) a mass spectrometer configured to detect one or more molecular masses of amplicons produced using at least one purified oligonucleotide primer pair that comprises forward and reverse primers about 15 to 35 nucleobases in length, wherein the forward primer comprises at least 70% (e.g., 70% . . . 75% . . . 90% . . . 95% . . . 100%) identity with a sequence selected from SEQ ID NOs:1-70, and wherein the reverse primer comprises at least 70% (e.g., 70% . . . 75% . . . 90% . . . 95% . . . 100%) identity with a sequence selected from SEQ ID NOs:71-140; and (b) a controller operably connected to the mass spectrometer, the controller configured to correlate the molecular masses of the amplicons with one or more species of fungi identities. In certain embodiments, the second primer pair is selected from the group of primer pair sequences consisting of: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, and 70:140, 141:142, 143:144, 145:146, 147:148, 149:150 and 151:152. In other embodiments, the controller is configured to determine base compositions of the amplicons from the molecular masses of the amplicons, which base compositions correspond to the one or more species and or sub-species classifications of fungi. In particular embodiments, the controller comprises or is operably connected to a database of known molecular masses and/or known base compositions of amplicons of known species and/or subspecies classifications of fungi produced with the primer pair.

In certain embodiments, the database comprises molecular mass information for at least three different bioagents. In other embodiments, the database comprises molecular mass information for at least 2 . . . 10 . . . 50 . . . 100 . . . 1000 . . . 10,000, or 100,000 different bioagents. In particular embodiments, the molecular mass information comprises base composition data. In some embodiments, the base composition data comprises at least 10 . . . 50 . . . 100 . . . 500 . . . 1000 . . . 1000 . . . 10,000 . . . or 100,000 unique base compositions. In other embodiments, the database comprises molecular mass information for a bioagent from two or more genera or species selected from the group consisting of Candida spp. (e.g., Candida albicans, Candida tropicalis, Candida glabrata, Candida parapsilosis and Candida crusei), Aspergillus spp., Fusarium spp. (e.g., Fusarium solani and Fusarium oxysporum), Cryptococus spp. (e.g., Cryptococcus neoformans), Coccidiodes spp. (e.g., Coccidiodes immitis, Coccidiodes posadasii), Mucor spp., Rhizopus spp., Mucorales (e.g., Mucoraceae, Ajellomyces spp.), and Pneumocystis jirovecii. In some embodiments, the database comprises molecular mass information for a bioagent from each of the genera or species Candida spp. (e.g., Candida albicans, Candida tropicalis, Candida glabrata, Candida parapsilosis and Candida crusei), Aspergillus spp., Fusarium spp. (e.g., Fusarium solani and Fusarium oxysporum), Cryptococus spp. (e.g., Cryptococcus neoformans), Coccidiodes spp. (e.g., Coccidiodes immitis, Coccidiodes posadasii), Mucor spp., Rhizopus spp., Mucorales (e.g., Mucoraceae, Ajellomyces spp.), and Pneumocystis jirovecii. In further embodiments, the database comprises molecular mass information for a fungal bioagent. In further embodiments, the database is stored on a local computer. In particular embodiments, the database is accessed from a remote computer over a network. In further embodiments, the molecular mass in the database is associated with bioagent identity. In certain embodiments, the molecular mass in the database is associated with bioagent geographic origin. In particular embodiments, bioagent identification comprises interrogation of the database with two or more different molecular masses (e.g., 2, 3, 4, 5, . . . 10 . . . 25 or more molecular masses) associated with the bioagent.

In some embodiments, the present invention provides compositions comprising at least one purified oligonucleotide primer 15 to 35 nucleobases in length, wherein the oligonucleotide primer comprises at least 70% (e.g., 70% . . . 75% . . . 90% . . . 95% . . . 100%) identity with a sequence selected from SEQ ID NOs: 1-152

### BRIEF DESCRIPTION OF THE DRAWINGS

The foregoing summary and detailed description is better understood when read in conjunction with the accompanying drawings which are included by way of example and not by way of limitation.

FIG. 1 shows a process diagram illustrating one embodiment of the primer pair selection process.

FIG. 2 shows a process diagram illustrating one embodiment of the primer pair validation process. Here select primers are shown meeting test criteria. Criteria include but are not limited to, the ability to amplify targeted fungi nucleic acid, the ability to exclude non-target bioagents, the ability to not produce unexpected amplicons, the ability to not dimerize, the ability to have analytical limits of detection of ≦100 genomic copies/reaction, and the ability to differentiate amongst different target organisms.

FIG. 3 shows a process diagram illustrating an embodiment of the calibration method.

FIG. 4 shows a block diagram showing a representative system.

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, about 200 nucleotides refers to a range encompassing between 180 and 220 nucleotides.

As used herein, the term “amplicon” or “bioagent identifying amplicon” refers to a nucleic acid generated using the primer pairs described herein. The amplicon is typically double stranded DNA; however, it may be RNA and/or DNA:RNA. In some embodiments, the amplicon comprises DNA complementary to fungal RNA, DNA, or cDNA. In some embodiments, the amplicon comprises sequences of conserved regions/primer pairs and intervening variable region. As discussed herein, primer pairs are configured to generate amplicons from fungi nucleic acid. As such, the base composition of any given amplicon may include the primer pair, the complement of the primer pair, the conserved regions and the variable region from the bioagent that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used to generate the molecular mass data. Generally, the amplicon further comprises a length that is compatible with mass spectrometry analysis. Bioagent identifying amplicons generate base compositions that are preferably unique to the identity of a bioagent (e.g., a fungus).

Amplicons typically comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One of ordinary skill in the art will further appreciate that the above range is not an absolute limit to the length of an amplicon, but instead represents a preferred length range. Amplicon lengths falling outside of this range are also included herein so long as the amplicon is amenable to calculation of a base composition signature as herein described.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, “fungus nucleic acid” includes, but is not limited to, DNA, RNA, or DNA that has been obtained from fungal RNA, such as, for example, by performing a reverse transcription reaction. Fungal RNA can either be single-stranded (of positive or negative polarity) or double stranded.

As used herein, the term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill F et al. Polymerase recognition of synthetic oligodeoxyribonucleotides incorporating degenerate pyrimidine and purine bases. Proc Natl Acad Sci USA. 1998 Apr. 14; 95(8):4258-63), an acyclic nucleoside analog containing 5-nitroindazole (Van Aerschot et al., Nucleosides and Nucleotides, 1995, 14, 1053-1056), the purine analog 1-(2-deoxy-beta-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, 06-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. Herein the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As used herein, a “base composition probability cloud” is a representation of the diversity in base composition resulting from a variation in sequence that occurs among different isolates of a given species, family or genus. Base composition calculations for a plurality of amplicons are mapped on a pseudo four-dimensional plot. Related members in a family, genus or species typically cluster within this plot, forming a base composition probability cloud.

As used herein, the term “base composition signature” refers to the base composition generated by any one particular amplicon.

As used herein, a “bioagent” means any biological organism or component thereof or a sample containing a biological organism or component thereof, including microorganisms or infectious substances, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Still, a non-exhaustive list of bioagents includes: cells, cell lines, human clinical samples, mammalian blood samples, cell cultures, bacterial cells, viruses, viroids, fungi, protists, parasites, rickettsiae, protozoa, animals, mammals or humans. Samples may be alive, non-replicating or dead or in a vegetative state (for example, vegetative bacteria or spores). Preferably, the bioagent is a fungi such as Candida spp. (e.g., Candida albicans, Candida tropicalis, Candida glabrata, Candida parapsilosis and Candida crusei), Aspergillus spp., Fusarium spp. (e.g., Fusarium solani and Fusarium oxysporum), Cryptococus spp. (e.g., Cryptococcus neoformans), Coccidiodes spp. (e.g., Coccidiodes immitis, Coccidiodes posadasii), Mucor spp., Rhizopus spp., Mucorales (e.g., Mucoraceae, Ajellomyces spp.), and Pneumocystis jirovecii.

As used herein, a “bioagent division” is defined as group of bioagents above the species level and includes but is not limited to, orders, families, genus, classes, clades, genera or other such groupings of bioagents above the species level.

As used herein, “broad range survey primers” are primers designed to identify an unknown bioagent as a member of a particular biological division (e.g., an order, family, class, clade, or genus). However, in some cases the broad range survey primers are also able to identify unknown bioagents at the species or sub-species level. As used herein, “division-wide primers” are primers designed to identify a bioagent at the species level and “drill-down” primers are primers designed to identify a bioagent at the sub-species level. As used herein, the “sub-species” level of identification includes, but is not limited to, strains, subtypes, variants, and isolates. Drill-down primers are not always required for identification at the sub-species level because broad range survey intelligent primers may, in some cases provide sufficient identification resolution to accomplishing this identification objective.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “conserved region” in the context of nucleic acids refers to a nucleobase sequence (e.g., a subsequence of a nucleic acid, etc.) that is the same or similar in two or more different regions or segments of a given nucleic acid molecule (e.g., an intramolecular conserved region), or that is the same or similar in two or more different nucleic acid molecules (e.g., an intermolecular conserved region). To illustrate, a conserved region may be present in two or more different taxonomic ranks (e.g., two or more different genera, two or more different species, two or more different subspecies, and the like) or in two or more different nucleic acid molecules from the same organism. To further illustrate, in certain embodiments, nucleic acids comprising at least one conserved region typically have between about 70%-100%, between about 80-100%, between about 90-100%, between about 95-100%, or between about 99-100% sequence identity in that conserved region. A conserved region may also be selected or identified functionally as a region that permits generation of amplicons via primer extension through hybridization of a completely or partially complementary primer to the conserved region for each of the target sequences to which conserved region is conserved.

The term “correlates” refers to establishing a relationship between two or more things. In certain embodiments, for example, detected molecular masses of one or more amplicons indicate the presence or identity of a given bioagent in a sample. In some embodiments, base compositions are calculated or otherwise determined from the detected molecular masses of amplicons, which base compositions indicate the presence or identity of a given bioagent in a sample.

As used herein, in some embodiments the term “database” is used to refer to a collection of base composition molecular mass data. In other embodiments the term “database” is used to refer to a collection of base composition data. The base composition data in the database is indexed to bioagents and to primer pairs. The base composition data reported in the database comprises the number of each nucleoside in an amplicon that would be generated for each bioagent using each primer. The database can be populated by empirical data. In this aspect of populating the database, a bioagent is selected and a primer pair is used to generate an amplicon. The amplicon's molecular mass is determined using a mass spectrometer and the base composition calculated therefrom without sequencing i.e., without determining the linear sequence of nucleobases comprising the amplicon. Note that base composition entries in the database may be derived from sequencing data (i.e., known sequence information), but the base composition of the amplicon to be identified is determined without sequencing the amplicon. An entry in the database is made to associate correlate the base composition with the bioagent and the primer pair used. The database may also be populated using other databases comprising bioagent information. For example, using the GenBank database it is possible to perform electronic PCR using an electronic representation of a primer pair. This in silico method may provide the base composition for any or all selected bioagent(s) stored in the GenBank database. The information may then be used to populate the base composition database as described above. A base composition database can be in silico, a written table, a reference book, a spreadsheet or any form generally amenable to databases. Preferably, it is in silico on computer readable media.

The term “detect”, “detecting” or “detection” refers to an act of determining the existence or presence of one or more targets (e.g., bioagent nucleic acids, amplicons, etc.) in a sample.

As used herein, the term “etiology” refers to the causes or origins, of diseases or abnormal physiological conditions.

As used herein, the term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length sequence or fragment thereof are retained.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to nucleic acid sequences that are not found naturally associated with the gene sequences in the chromosome or are associated with portions of the chromosome not found in nature (e.g., genes expressed in loci where the gene is not normally expressed).

The terms “homology,” “homologous” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/20=0.75 or 75% sequence identity with the 20 nucleobase primer. In context of the present invention, sequence identity is meant to be properly determined when the query sequence and the subject sequence are both described and aligned in the 5′ to 3′ direction. Sequence alignment algorithms such as BLAST, will return results in two different alignment orientations. In the Plus/Plus orientation, both the query sequence and the subject sequence are aligned in the 5′ to 3′ direction. On the other hand, in the Plus/Minus orientation, the query sequence is in the 5′ to 3′ direction while the subject sequence is in the 3′ to 5′ direction. It should be understood that with respect to the primers of the present invention, sequence identity is properly determined when the alignment is designated as Plus/Plus. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, “housekeeping gene” or “core viral gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (Tₘ) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

As used herein, “intelligent primers” or “primers” or “primer pairs,” in some embodiments, are oligonucleotides that are designed to bind to conserved sequence regions of one or more bioagent nucleic acids to generate bioagent identifying amplicons. In some embodiments, the bound primers flank an intervening variable region between the conserved binding sequences. Upon amplification, the primer pairs yield amplicons e.g., amplification products that provide base composition variability between the two or more bioagents. The variability of the base compositions allows for the identification of one or more individual bioagents from, e.g., two or more bioagents based on the base composition distinctions. In some embodiments, the primer pairs are also configured to generate amplicons amenable to molecular mass analysis. Further, the sequences of the primer members of the primer pairs are not necessarily fully complementary to the conserved region of the reference bioagent. For example, in some embodiments, the sequences are designed to be “best fit” amongst a plurality of bioagents at these conserved binding sequences. Therefore, the primer members of the primer pairs have substantial complementarity with the conserved regions of the bioagents, including the reference bioagent.

In some embodiments of the invention, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J. Am. Chem. Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more fungi. Samples can include, for example, evidence from a crime scene, blood, blood stains, sputum, nasal swabs, throat swabs, skin scrapings, fungal and yeast culture isolates, bronchoalveolar lavage samples, transplanted tissue samples, direct tissue biopsy samples, semen, semen stains, bone, teeth, hair, saliva, urine, feces, fingernails, muscle tissue, cigarettes, stamps, envelopes, dandruff, fingerprints, personal items, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As is used herein, the term “single primer pair identification” means that one or more bioagents can be identified using a single primer pair. A base composition signature for an amplicon may singly identify one or more bioagents.

As used herein, a “sub-species characteristic” is a genetic characteristic that provides the means to distinguish two members of the same bioagent species. For example, one fungal strain may be distinguished from another fungal strain of the same species by possessing a genetic change (e.g., for example, a nucleotide deletion, addition or substitution) in one of the fungal genes, such as the RNA-dependent RNA polymerase.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a nucleic acid from a given bioagent. Similarly, the primer pairs provided herein may comprise between about 70%-100%, or between about 80-100%, or between about 90-100%, or between about 95-100% identity, or between about 99-100% sequence identity with the primer pairs disclosed in Table 1. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. In some embodiments, any oligonucleotide primer pair may have one or both primers with less then 70% sequence homology with a corresponding member of any of the primer pairs of Table 1 if the primer pair has the capability of producing an amplification product corresponding to the desired fungi identifying amplicon.

A “system” in the context of analytical instrumentation refers a group of objects and/or devices that form a network for performing a desired objective.

As used herein, “triangulation identification” means the use of more than one primer pair to generate a corresponding amplicon for identification of a bioagent. The more than one primer pair can be used in individual wells or vessels or in a multiplex PCR assay. Alternatively, PCR reactions may be carried out in single wells or vessels comprising a different primer pair in each well or vessel. Following amplification the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation is a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process may also be used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected compositions from the B. anthracis genome would suggest a genetic engineering event.

As used herein, the term “unknown bioagent” can mean, for example: (i) a bioagent whose existence is not known (for example, the SARS coronavirus was unknown prior to April 2003) and/or (ii) a bioagent whose existence is known (such as the well known bacterial species Staphylococcus aureus for example) but which is not known to be in a sample to be analyzed. For example, if the method for identification of coronaviruses disclosed in commonly owned U.S. patent Ser. No. 10/829,826 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. patent Ser. No. 10/829,826 was to be employed subsequent to April 2003 to identify the SARS coronavirus in a clinical sample, the second meaning (ii) of “unknown” bioagent would apply because the SARS coronavirus became known to science subsequent to April 2003 because it was not known what bioagent was present in the sample.

As used herein, the term “variable region” is used to describe a region that falls between any one primer pair described herein. The region possesses distinct base compositions between at least two bioagents, such that at least one bioagent can be identified at, for example, the family, genus, species or sub-species level. The degree of variability between the at least two bioagents need only be sufficient to allow for identification using mass spectrometry analysis, as described herein.

As used herein, a “wobble base” is a variation in a codon found at the third nucleotide position of a DNA triplet. Variations in conserved regions of sequence are often found at the third nucleotide position due to redundancy in the amino acid code.

Provided herein are methods, compositions, kits, and related systems for the detection and identification of bioagents (e.g., genera or species of fungi) using bioagent identifying amplicons. In some embodiments, the present invention provides assays comprising primers for broad range detection of fungi genera and specific detection of clinically relevant fungi at the species or other level.

In some embodiments, primers are selected to hybridize to conserved sequence regions of nucleic acids derived from a bioagent and which flank variable sequence regions to yield a bioagent identifying amplicon which can be amplified and which is amenable to molecular mass determination. In some embodiments, the molecular mass is converted to a base composition, which indicates the number of each nucleotide in the amplicon. Systems employing software and hardware useful in converting molecular mass data into base composition information are available from, for example, Ibis Biosciences, Inc. (Carlsbad, Calif.), for example the Ibis T5000 Biosensor System, and are described in U.S. patent application Ser. No. 10/754,415, filed Jan. 9, 2004, incorporated by reference herein in its entirety. In some embodiments, the molecular mass or corresponding base composition of one or more different amplicons is queried against a database of molecular masses or base compositions indexed to bioagents and to the primer pair used to generate the amplicon. A match of the measured base composition to a database entry base composition associates the sample bioagent to an indexed bioagent in the database. Thus, the identity of the unknown bioagent is determined. No prior knowledge of the unknown bioagent is necessary to make an identification. In some instances, the measured base composition associates with more than one database entry base composition. Thus, a second/subsequent primer pair is generally used to generate an amplicon, and its measured base composition is similarly compared to the database to determine its identity in triangulation identification. Furthermore, the methods and other aspects of the invention can be applied to rapid parallel multiplex analyses, the results of which can be employed in a triangulation identification strategy. Thus, in some embodiments, the present invention provides rapid throughput and does not require nucleic acid sequencing or knowledge of the linear sequences of nucleobases of the amplified target sequence for bioagent detection and identification.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; WO2002/070664; WO2003/001976; WO2003/100035; WO2004/009849; WO2004/052175; WO2004/053076; WO2004/053141; WO2004/053164; WO2004/060278; WO2004/093644; WO2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222; Ecker et al., Ibis T5000: a universal biosensor approach for microbiology. Nat Rev Microbiol. 2008 Jun. 3.; Ecker et al., The Microbial Rosetta Stone Database: A compilation of global and emerging infectious microorganisms and bioterrorist threat agents. BMC Microbiology. 2005. 5(1): 19.; Ecker et al., The Ibis T5000 Universal Biosensor: An Automated Platform for

Pathogen Identification and Strain Typing. JALA. 2006. 6(11): 341-351.; Ecker et al., The Microbial Rosetta Stone Database: A common structure for microbial biosecurity threat agents. J Forensic Sci. 2005. 50(6): 1380-5.; Ecker et al., Identification of Acinetobacter species and genotyping of Acinetobacter baumannii by multilocus PCR and mass spectrometry. J Clin Microbiol. 2006 August; 44(8):2921-32.; Ecker et al., Rapid identification and strain-typing of respiratory pathogens for epidemic surveillance. Proc Natl Acad Sci USA. 2005 May 31; 102(22):8012-7. Epub 2005 May 23.; Wortmann et al., Genotypic Evolution of Acinetobacter baumannii Strains in an Outbreak Associated With War Trauma. Infect Control Hosp Epidemiol. 2008 June; 29(6):553-555.; Hannis et al., High-resolution genotyping of Campylobacter species by use of PCR and high-throughput mass spectrometry. J Clin Microbiol. 2008 April; 46(4):1220-5.; Blyn et al., Rapid detection and molecular serotyping of adenovirus by use of PCR followed by electrospray ionization mass spectrometry. J Clin Microbiol. 2008 February; 46(2):644-51.; Eshoo et al., Direct broad-range detection of alphaviruses in mosquito extracts. Virology. 2007 Nov. 25; 368(2):286-95.; Sampath et al., Global surveillance of emerging Influenza virus genotypes by mass spectrometry. PLoS ONE. 2007 May 30; 2(5):e489.; Sampath et al., Rapid identification of emerging infectious agents using PCR and electrospray ionization mass spectrometry. Ann NY Acad Sci. 2007 April; 1102:109-20.; Hujer et al., Analysis of antibiotic resistance genes in multidrug-resistant Acinetobacter sp. isolates from military and civilian patients treated at the Walter Reed Army Medical Center. Antimicrob Agents Chemother. 2006 December; 50(12):4114-23.; Hall et al., Base composition analysis of human mitochondrial DNA using electrospray ionization mass spectrometry: a novel tool for the identification and differentiation of humans. Anal Biochem. 2005 Sep. 1; 344(1):53-69.; Sampath et al., Rapid identification of emerging pathogens: coronavirus. Emerg Infect Dis. 2005 March; 11(3):373-9.; Jiang Y, Hofstadler S A. A highly efficient and automated method of purifying and desalting PCR products for analysis by electrospray ionization mass spectrometry. Anal Biochem. 2003. 316: 50-57.; Jiang et al., Mitochondrial DNA mutation detection by electrospray mass spectrometry. Clin Chem. 2006. 53(2): 195-203. Epub December 7.; Russell et al., Transmission dynamics and prospective environmental sampling of adenovirus in a military recruit setting. J Infect Dis. 2006. 194(7): 877-85. Epub 2006 Aug. 25.; Hofstadler et al., Detection of microbial agents using broad-range PCR with detection by mass spectrometry: The TIGER concept. Chapter in Encyclopedia of Rapid Microbiological Methods. 2006.; Hofstadler et al., Selective ion filtering by digital thresholding: A method to unwind complex ESI-mass spectra and eliminate signals from low molecular weight chemical noise. Anal Chem. 2006. 78(2): 372-378.; Hofstadler et al., TIGER: The Universal Biosensor. Int J Mass Spectrom. 2005. 242(1): 23-41.; Van Ert et al., Mass spectrometry provides accurate characterization of two genetic marker types in Bacillus anthracis. Biotechniques. 2004. 37(4): 642-4, 646, 648.; Sampath et al., Forum on Microbial Threats: Learning from SARS: Preparing for the Next Disease Outbreak—Workshop Summary. (ed. Knobler S E, Mahmoud A, Lemon S.) The National Academies Press, Washington, D.C. 2004. 181-185.

In certain embodiments, bioagent identifying amplicons amenable to molecular mass determination produced by the primers described herein are either of a length, size or mass compatible with a particular mode of molecular mass determination, or compatible with a means of providing a fragmentation pattern in order to obtain fragments of a length compatible with a particular mode of molecular mass determination. Such means of providing a fragmentation pattern of an amplicon include, but are not limited to, cleavage with restriction enzymes or cleavage primers, sonication or other means of fragmentation. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR). Other amplification methods may be used such as ligase chain reaction (LCR), low-stringency single primer PCR, and multiple strand displacement amplification (MDA). (Michael, S F., Biotechniques (1994), 16:411-412 and Dean et al., Proc. Natl. Acad. Sci. U.S.A. (2002), 99, 5261-5266).

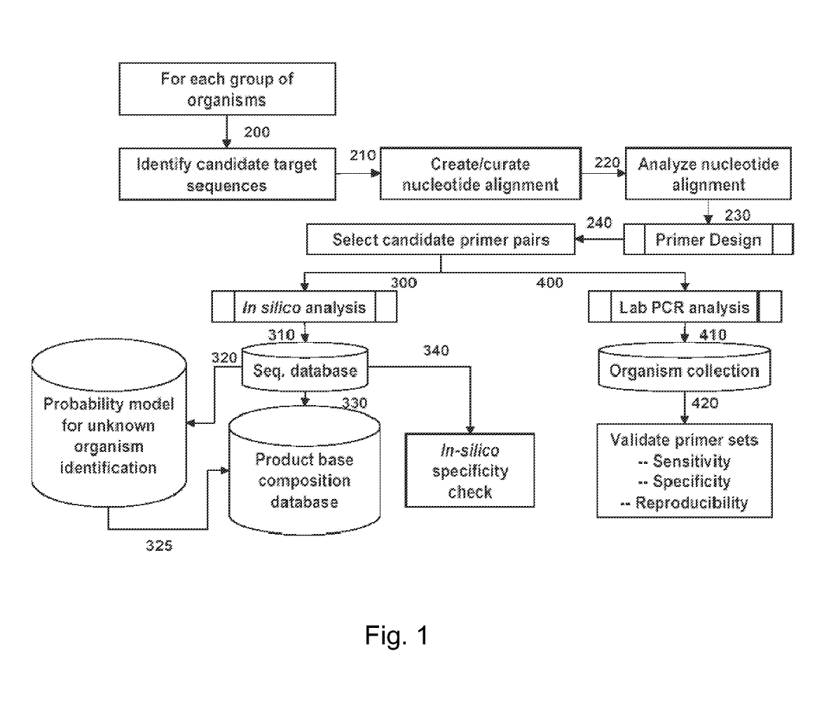

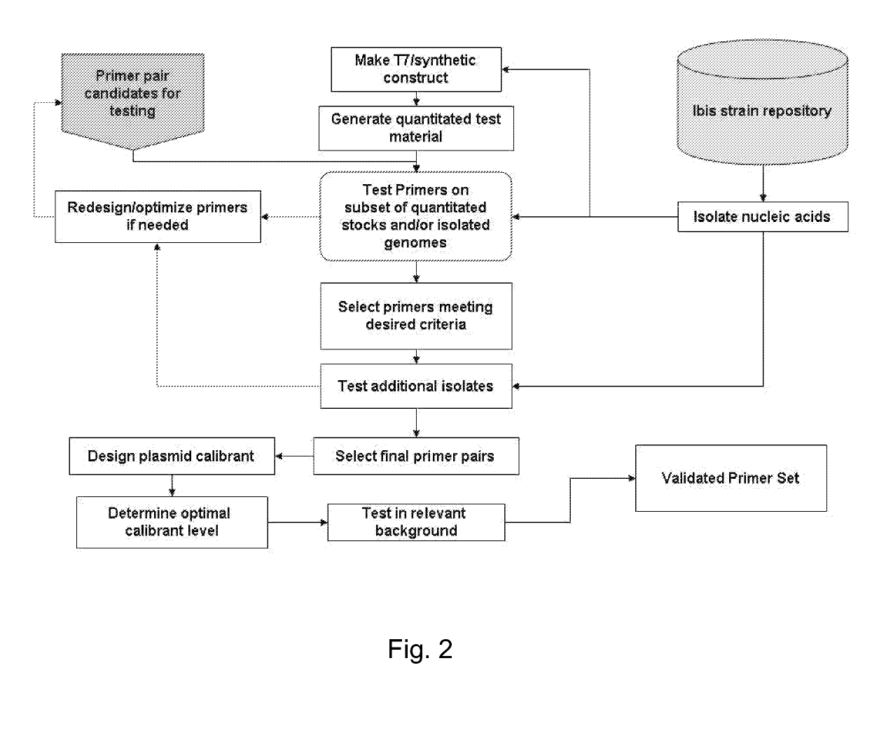

One embodiment of a process flow diagram used for primer selection and validation process is depicted in FIGS. 1 and 2. For each group of organisms, candidate target sequences are identified (200) from which nucleotide sequence alignments are created (210) and analyzed (220). Primers are then configured by selecting priming regions (230) to facilitate the selection of candidate primer pairs (240). The primer pair sequence is typically a “best fit” amongst the aligned sequences, such that the primer pair sequence may or may not be fully complementary to the hybridization region on any one of the bioagents in the alignment. Thus, best fit primer pair sequences are those with sufficient complementarity with two or more bioagents to hybridize with the two or more bioagents and generate an amplicon. The primer pairs are then subjected to in silico analysis by electronic PCR (ePCR) (300) wherein bioagent identifying amplicons are obtained from sequence databases such as GenBank or other sequence collections (310) and tested for specificity in silico (320). Bioagent identifying amplicons obtained from ePCR of GenBank sequences (310) may also be analyzed by a probability model which predicts the capability of a given amplicon to identify unknown bioagents. Preferably, the base compositions of amplicons with favorable probability scores are then stored in a base composition database (325). Alternatively, base compositions of the bioagent identifying amplicons obtained from the primers and GenBank sequences are directly entered into the base composition database (330). Candidate primer pairs (240) are validated by in vitro amplification by a method such as PCR analysis (400) of nucleic acid from a collection of organisms (410). Amplicons thus obtained are analyzed to confirm the sensitivity, specificity and reproducibility of the primers used to obtain the amplicons (420).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers typically are employed as compositions for use in methods for identification of bioagents as follows: a primer pair composition is contacted with nucleic acid (such as, for example, DNA) of an unknown isolate suspected of comprising a genus or species of fungi. The nucleic acid is then amplified by a nucleic acid amplification technique, such as PCR for example, to obtain an amplicon that represents a bioagent identifying amplicon. The molecular mass of the strands of the double-stranded amplicon is determined by a molecular mass measurement technique such as mass spectrometry, for example. Preferably the two strands of the double-stranded amplicon are separated during the ionization process; however, they may be separated prior to mass spectrometry measurement. In some embodiments, the mass spectrometer is electrospray Fourier transform ion cyclotron resonance mass spectrometry (ESI-FTICR-MS) or electrospray time of flight mass spectrometry (ESI-TOF-MS). A list of possible base compositions may be generated for the molecular mass value obtained for each strand, and the choice of the base composition from the list is facilitated by matching the base composition of one strand with a complementary base composition of the other strand. A measured molecular mass or base composition calculated therefrom is then compared with a database of molecular masses or base compositions indexed to primer pairs and to known bioagents. A match between the measured molecular mass or base composition of the amplicon and the database molecular mass or base composition for that indexed primer pair correlates the measured molecular mass or base composition with an indexed bioagent, thus identifying the unknown bioagent (e.g. the genus or species of fungi). In some embodiments, the primer pair used is at least one of the primer pairs of Table 1. In some embodiments, the method is repeated using a different primer pair to resolve possible ambiguities in the identification process or to improve the confidence level for the identification assignment (triangulation identification). In some embodiments, for example, where the unknown is a novel, previously uncharacterized organism, the molecular mass or base composition from an amplicon generated from the unknown is matched with one or more best match molecular masses or base compositions from a database to predict a family, genus, species, sub-type, etc. of the unknown. Such information may assist further characterization of the unknown or provide a physician treating a patient infected by the unknown with a therapeutic agent best calculated to treat the patient.

In certain embodiments, fungi are detected with the systems and methods of the present invention in combination with other bioagents, including viruses, bacteria, fungi, or other bioagents. In particular embodiments, a panel is employed that includes fungi and other related or un-related bioagents. Such panels may be specific for a particular type of bioagent, or specific for a specific type of test (e.g., for testing the safety of blood, one may include commonly present viral pathogens such as HCV, HIV, and bacteria that can be contracted via a blood transfusion).

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR).

In some embodiments, the oligonucleotide primers are broad range survey primers which hybridize to conserved regions of nucleic acid. The broad range primer may identify the unknown bioagent depending on which bioagent is in the sample. In other cases, the molecular mass or base composition of an amplicon does not provide sufficient resolution to identify the unknown bioagent as any one bioagent at or below the species level. These cases generally benefit from further analysis of one or more amplicons generated from at least one additional broad range survey primer pair, or from at least one additional division-wide primer pair, or from at least one additional drill-down primer pair. Identification of sub-species characteristics may be required, for example, to determine a clinical treatment of patient, or in rapidly responding to an outbreak of a new species, sub-type, etc. of pathogen to prevent an epidemic or pandemic.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus they need not be fully complementary to the hybridization region of any one of the bioagents in the alignment. Moreover, a primer may hybridize over one or more segments such that intervening or adjacent segments are not involved in the hybridization event (e.g., for example, a loop structure or a hairpin structure). The primers may comprise at least 70%, at least 75%, at least 80%, at least 85%, at least 90%, at least 95% or at least 99% sequence identity with any of the primers listed in Table 1. Thus, in some embodiments, an extent of variation of 70% to 100%, or any range falling within, of the sequence identity is possible relative to the specific primer sequences disclosed herein. To illustrate, determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions of viral nucleic acid, is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq DNA polymerase (Magnuson et al., Biotechniques, 1996, 21, 700-709), an occurrence which may lead to ambiguous results arising from molecular mass analysis.

Primers may contain one or more universal bases. Because any variation (due to codon wobble in the third position) in the conserved regions among species is likely to occur in the third position of a DNA (or RNA) triplet, oligonucleotide primers can be designed such that the nucleotide corresponding to this position is a base which can bind to more than one nucleotide, referred to herein as a “universal nucleobase.” For example, under this “wobble” base pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003.), the degenerate nucleotides dP or dK, an acyclic nucleoside analog containing 5-nitroindazole (Van Aerschot et al., Nucleosides and Nucleotides., 1995, 14, 1053-1056.) or the purine analog 1-(2-deoxy-beta-D-ribofuranosyl)-imidazole-4-carboxamide (Sala et al., Nucl Acids Res., 1996, 24, 3302-3306).

In some embodiments, to compensate for weaker binding by the wobble base, oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S Pre-Grant Publication No. 2003-0170682; also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in the determination of base composition of amplicons. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹³N and ¹³C.

In some embodiments, the molecular mass of a given bioagent (e.g., a genus or species of fungi) identifying amplicon is determined by mass spectrometry. Mass spectrometry is intrinsically a parallel detection scheme without the need for radioactive or fluorescent labels, because an amplicon is identified by its molecular mass. The current state of the art in mass spectrometry is such that less than femtomole quantities of material can be analyzed to provide information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, may vary slightly from strain to strain within species, for example. In some embodiments, the pattern classifier model is the mutational probability model. In other embodiments, the pattern classifier is the polytope model. A polytope model is the mutational probability model that incorporates both the restrictions among strains and position dependence of a given nucleobase within a triplet. In certain embodiments, a polytope pattern classifier is used to classify a test or unknown organism according to its amplicon base composition.

In some embodiments, it is possible to manage this diversity by building “base composition probability clouds” around the composition constraints for each species. A “pseudo four-dimensional plot” may be used to visualize the concept of base composition probability clouds. Optimal primer design typically involves an optimal choice of bioagent identifying amplicons and maximizes the separation between the base composition signatures of individual bioagents. Areas where clouds overlap generally indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition has not been previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein is bioagent classifying information at a level sufficient to identify a given bioagent. Furthermore, the process of determining a previously unknown base composition for a given bioagent (for example, in a case where sequence information is unavailable) has utility by providing additional bioagent indexing information with which to populate base composition databases. The process of future bioagent identification is thus improved as additional base composition signature indexes become available in base composition databases.

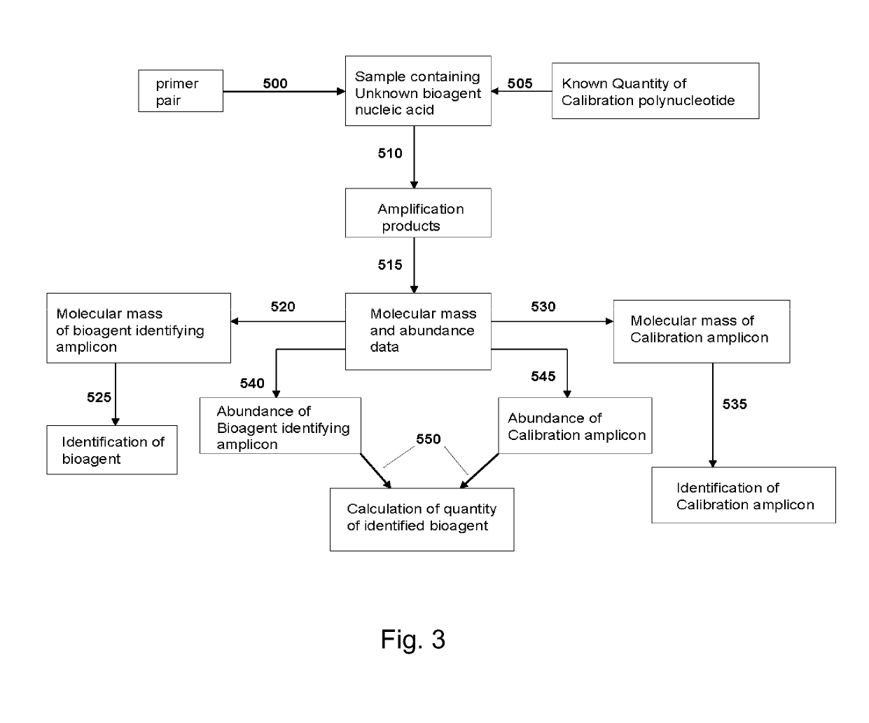

In some embodiments, the identity and quantity of an unknown bioagent may be determined using the process illustrated in FIG. 3. Primers (500) and a known quantity of a calibration polynucleotide (505) are added to a sample containing nucleic acid of an unknown bioagent. The total nucleic acid in the sample is then subjected to an amplification reaction (510) to obtain amplicons. The molecular masses of amplicons are determined (515) from which are obtained molecular mass and abundance data. The molecular mass of the bioagent identifying amplicon (520) provides for its identification (525) and the molecular mass of the calibration amplicon obtained from the calibration polynucleotide (530) provides for its quantification (535). The abundance data of the bioagent identifying amplicon is recorded (540) and the abundance data for the calibration data is recorded (545), both of which are used in a calculation (550) which determines the quantity of unknown bioagent in the sample.

In certain embodiments, a sample comprising an unknown bioagent is contacted with a primer pair which amplifies the nucleic acid from the bioagent, and a known quantity of a polynucleotide that comprises a calibration sequence. The amplification reaction then produces two amplicons: a bioagent identifying amplicon and a calibration amplicon. The bioagent identifying amplicon and the calibration amplicon are distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve in which the amount of calibration or calibrant polynucleotide spiked into the sample is varied provides additional resolution and improved confidence for the determination of the quantity of bioagent in the sample. Alternatively, the calibration polynucleotide can be amplified in its own reaction vessel or vessels under the same conditions as the bioagent. A standard curve may be prepared there from, and the relative abundance of the bioagent determined by methods such as linear regression. In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single construct (preferably a vector) which functions as the calibration polynucleotide.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide gives rise to a calibration amplicon. Failure to produce a measurable calibration amplicon indicates a failure of amplification or subsequent analysis step such as amplicon purification or molecular mass determination. Reaching a conclusion that such failures have occurred is, in itself, a useful event. In some embodiments, the calibration sequence is comprised of DNA. In some embodiments, the calibration sequence is comprised of RNA.

In some embodiments, a calibration sequence is inserted into a vector which then functions as the calibration polynucleotide. In some embodiments, more than one calibration sequence is inserted into the vector that functions as the calibration polynucleotide. Such a calibration polynucleotide is herein termed a “combination calibration polynucleotide.” It should be recognized that the calibration method should not be limited to the embodiments described herein. The calibration method can be applied for determination of the quantity of any bioagent identifying amplicon when an appropriate standard calibrant polynucleotide sequence is designed and used.

In certain embodiments, primer pairs are configured to produce bioagent identifying amplicons within more conserved regions of an fungi, while others produce bioagent identifying amplicons within regions that are may evolve more quickly. Primer pairs that characterize amplicons in a conserved region with low probability that the region will evolve past the point of primer recognition are useful, e.g., as a broad range survey-type primer. Primer pairs that characterize an amplicon corresponding to an evolving genomic region are useful, e.g., for distinguishing emerging bioagent strain variants.

The primer pairs described herein provide reagents, e.g., for identifying diseases caused by emerging genera, species, strains or types of fungi. Base composition analysis eliminates the need for prior knowledge of bioagent sequence to generate hybridization probes. Thus, in another embodiment, there is provided a method for determining the etiology of a particular stain when the process of identification of is carried out in a clinical setting, and even when a new strain is involved. This is possible because the methods may not be confounded by naturally occurring evolutionary variations.

Another embodiment provides a means of tracking the spread of any species or strain of fungi when a plurality of samples obtained from different geographical locations are analyzed by methods described above in an epidemiological setting. For example, a plurality of samples from a plurality of different locations may be analyzed with primers which produce bioagent identifying amplicons, a subset of which identifies a specific strain. The corresponding locations of the members of the strain-containing subset indicate the spread of the specific strain to the corresponding locations.

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to twenty primer pairs, from one to ten primer pairs, from one to eight pairs, from one to five primer pairs, from one to three primer pairs, or from one to two primer pairs. In some embodiments, the kit may comprise one or more primer pairs recited in Table 1. In certain embodiments, the kits include all of the primer pairs recited in Table 2.

In some embodiments, the kit may also comprise a sufficient quantity of reverse transcriptase, a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. In some embodiments, the kit further comprises instructions for analysis, interpretation and dissemination of data acquired by the kit. In other embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for isolating bioagent nucleic acid or bioagent identifying amplicons from amplification reactions, including, for example, detergents, solvents, or ion exchange resins which may be linked to magnetic beads. A kit may also comprise a table of measured or calculated molecular masses and/or base compositions of bioagents using the primer pairs of the kit.

The invention also provides systems that can be used to perform various assays relating to fungi detection or identification. In certain embodiments, systems include mass spectrometers configured to detect molecular masses of amplicons produced using purified oligonucleotide primer pairs described herein. Other detectors that are optionally adapted for use in the systems of the invention are described further below. In some embodiments, systems also include controllers operably connected to mass spectrometers and/or other system components. In some of these embodiments, controllers are configured to correlate the molecular masses of the amplicons with bioagents to effect detection or identification. In some embodiments, controllers are configured to determine base compositions of the amplicons from the molecular masses of the amplicons. As described herein, the base compositions generally correspond to the fungi species identities. In certain embodiments, controllers include, or are operably connected to, databases of known molecular masses and/or known base compositions of amplicons of known species of fungi produced with the primer pairs described herein. Controllers are described further below.

In some embodiments, systems include one or more of the primer pairs described herein (e.g., in Table 1). In certain embodiments, the oligonucleotides are arrayed on solid supports, whereas in others, they are provided in one or more containers, e.g., for assays performed in solution. In certain embodiments, the systems also include at least one detector or detection component (e.g., a spectrometer) that is configured to detect detectable signals produced in the container or on the support. In addition, the systems also optionally include at least one thermal modulator (e.g., a thermal cycling device) operably connected to the containers or solid supports to modulate temperature in the containers or on the solid supports, and/or at least one fluid transfer component (e.g., an automated pipettor) that transfers fluid to and/or from the containers or solid supports, e.g., for performing one or more assays (e.g., nucleic acid amplification, real-time amplicon detection, etc.) in the containers or on the solid supports.

Detectors are typically structured to detect detectable signals produced, e.g., in or proximal to another component of the given assay system (e.g., in a container and/or on a solid support). Suitable signal detectors that are optionally utilized, or adapted for use, herein detect, e.g., fluorescence, phosphorescence, radioactivity, absorbance, refractive index, luminescence, or mass. Detectors optionally monitor one or a plurality of signals from upstream and/or downstream of the performance of, e.g., a given assay step. For example, detectors optionally monitor a plurality of optical signals, which correspond in position to “real-time” results. Example detectors or sensors include photomultiplier tubes, CCD arrays, optical sensors, temperature sensors, pressure sensors, pH sensors, conductivity sensors, or scanning detectors. Detectors are also described in, e.g., Skoog et al., Principles of Instrumental Analysis, 5^{th} Ed., Harcourt Brace College Publishers (1998), Currell, Analytical Instrumentation: Performance Characteristics and Quality, John Wiley & Sons, Inc. (2000), Sharma et al., Introduction to Fluorescence Spectroscopy, John Wiley & Sons, Inc. (1999), Valeur, Molecular Fluorescence: Principles and Applications, John Wiley & Sons, Inc. (2002), and Gore, Spectrophotometry and Spectrofluorimetry: A Practical Approach, 2.sup.nd Ed., Oxford University Press (2000), which are each incorporated by reference.

As mentioned above, the systems of the invention also typically include controllers that are operably connected to one or more components (e.g., detectors, databases, thermal modulators, fluid transfer components, robotic material handling devices, and the like) of the given system to control operation of the components. More specifically, controllers are generally included either as separate or integral system components that are utilized, e.g., to receive data from detectors (e.g., molecular masses, etc.), to effect and/or regulate temperature in the containers, or to effect and/or regulate fluid flow to or from selected containers. Controllers and/or other system components are optionally coupled to an appropriately programmed processor, computer, digital device, information appliance, or other logic device (e.g., including an analog to digital or digital to analog converter as needed), which functions to instruct the operation of these instruments in accordance with preprogrammed or user input instructions, receive data and information from these instruments, and interpret, manipulate and report this information to the user. Suitable controllers are generally known in the art and are available from various commercial sources.

Any controller or computer optionally includes a monitor, which is often a cathode ray tube (“CRT”) display, a flat panel display (e.g., active matrix liquid crystal display or liquid crystal display), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. These components are illustrated further below.

The computer typically includes appropriate software for receiving user instructions, either in the form of user input into a set of parameter fields, e.g., in a graphic user interface (GUI), or in the form of preprogrammed instructions, e.g., preprogrammed for a variety of different specific operations. The software then converts these instructions to appropriate language for instructing the operation of one or more controllers to carry out the desired operation. The computer then receives the data from, e.g., sensors/detectors included within the system, and interprets the data, either provides it in a user understood format, or uses that data to initiate further controller instructions, in accordance with the programming.

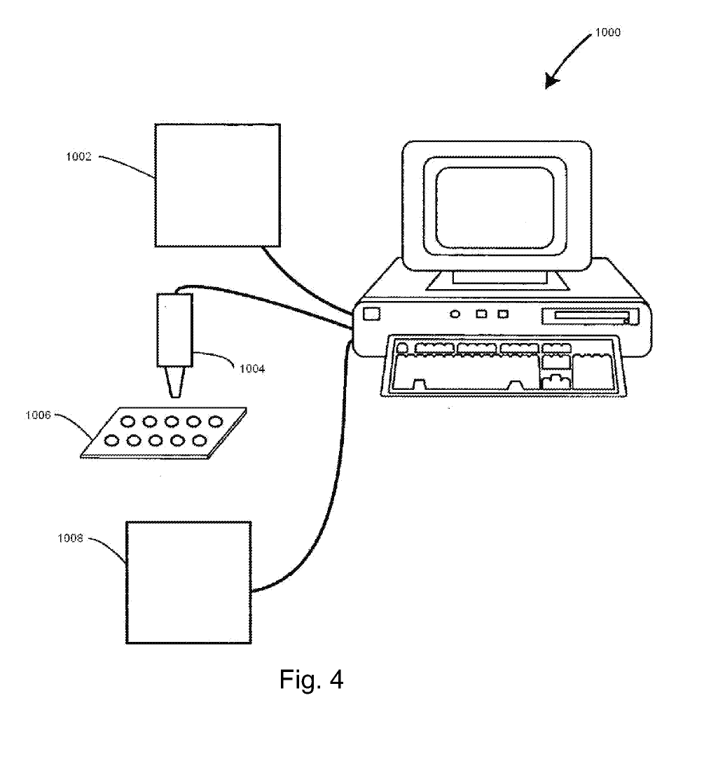

FIG. 4 is a schematic showing a representative system that includes a logic device in which various aspects of the present invention may be embodied. As will be understood by practitioners in the art from the teachings provided herein, aspects of the invention are optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform as desired. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

More specifically, FIG. 4 schematically illustrates computer 1000 to which mass spectrometer 1002 (e.g., an ESI-TOF mass spectrometer, etc.), fluid transfer component 1004 (e.g., an automated mass spectrometer sample injection needle or the like), and database 1008 are operably connected. Optionally, one or more of these components are operably connected to computer 1000 via a server (not shown in FIG. 4). During operation, fluid transfer component 1004 typically transfers reaction mixtures or components thereof (e.g., aliquots comprising amplicons) from multi-well container 1006 to mass spectrometer 1002. Mass spectrometer 1002 then detects molecular masses of the amplicons. Computer 1000 then typically receives this molecular mass data, calculates base compositions from this data, and compares it with entries in database 1008 to identify genus, species or strains of fungi in a given sample. It will be apparent to one of skill in the art that one or more components of the system schematically depicted in FIG. 4 are optionally fabricated integral with one another (e.g., in the same housing).

While the present invention has been described with specificity in accordance with certain of its embodiments, the following examples serve only to illustrate the invention and are not intended to limit the same. In order that the invention disclosed herein may be more efficiently understood, examples are provided below. It should be understood that these examples are for illustrative purposes only and are not to be construed as limiting the invention in any manner.

### EXAMPLE 1

### High-Throughput ESI-Mass Spectrometry Assay for the Identification of Fungi

This example describes a fungi identification assay which employs mass spectrometry determined base compositions for PCR amplicons derived from various fungi. The T5000 Biosensor System is a mass spectrometry based universal biosensor that uses mass measurements to derived base compositions of PCR amplicons to identify bioagents including, for example, bacteria, fungi, viruses and protozoa (S. A. Hofstadler et. al. Int. J. Mass Spectrom. (2005) 242:23-41, herein incorporated by reference). For this fungal detection assay, broad range and specific primers were selected (Table 1). The base composition of the PCR amplicons can be determined and compared to a database of known fungi base compositions to determine the identity of a specific genus or species of fungi in a sample.

Shown below, in Table 1A, are the sequences for the both the forward and reverse primers for both broad and specific identification of fungi. Additional details relating to the primer pairs provided in Table 1A are provided in Tables 1B, 1C and Table 2.

TABLE 1A
Sequences of Primer Pairs

| Primer | Primer | forward primer | SEQ ID |
|---|---|---|---|
| pair | Direction | sequence | NO |
| 3029 | Forward | TCTCAGGATAGCAG | 1 |
| | | AAGCTCGTATCAG | |
| 3030 | Forward | TGTGAAGCGGCAA | 2 |
| | | AAGCTCAAATTT | |
| 3031 | Forward | TGGAGTCTAACAT | 3 |
| | | CTATGCGAGTGTT | |
| 3032 | Forward | TTGTAGAATAGGT | 4 |
| | | GGGAGCTTCGGC | |
| 3766 | Forward | TTGTGTAGAATAGG | 5 |
| | | TGGGAGCTTCGGC | |
| 3815 | Forward | TCGATGAAGAAC | 6 |
| | | GCAGCGAAATGC | |
| 3816 | Forward | TCGATGAAGAAC | 7 |
| | | GCAGCGAAATGC | |
| 3856 | Forward | TGGTCCTTGAAAATC | 8 |
| | | CACAGGAAGGAATAG | |
| 3857 | Forward | TCCCGTACGCGTA | 9 |
| | | ATGAAAGTGAACG | |
| 3858 | Forward | TGTCAAACCCGTACG | 10 |
| | | CGTAATGAAAGTGAA | |
| 3859 | Forward | TGATTCAGAGCTCC | 11 |
| | | AGAAGCTTTGTTC | |
| 3860 | Forward | TCGATCGACCAC | 12 |
| | | ATTGGGTCTGA | |
| 3861 | Forward | TCCTTCTATGTAAA | 13 |
| | | AACTGACGTTGAGG | |
| 3862 | Forward | TCCAAGTACTTGA | 14 |
| | | CACATGCTAATCG | |
| 3913 | Forward | TGGATTAGATACCCT | 15 |
| | | AGTAGTCCAAGCAG | |
| 3863 | Forward | TTTCATATATTTCGCACT | 16 |
| | | AATCACTCATCAATAGCA | |
| 3864 | Forward | TCGGATGAAATGCAA | 17 |
| | | GACATTAGACGGTTA | |
| 3865 | Forward | TGGTACAGTGGAGTA | 18 |
| | | TGCTGTTTAATTGGA | |
| 3866 | Forward | TGCGACATGGATT | 19 |
| | | CGGTACCCATTTA | |
| 3867 | Forward | TTCGATACCCGTGTA | 20 |
| | | GTTCTAGTAGTAAAC | |
| 3868 | Forward | TTCGATACCCGTGTA | 21 |
| | | GTTCTAGTAGTAAAC | |
| 3869 | Forward | TGTCCCAGGTGACA | 22 |
| | | ATGTTGGTTTCAAC | |
| 3870 | Forward | TGCCATTGCAAGATGTT | 23 |
| | | TACAAGATTGGTGGTAT | |
| 3871 | Forward | TAGAAGAGGTAACGTT | 24 |
| | | TGTGGTGACTCCAAG | |
| 3912 | Forward | TGTCCCAGGTGACA | 25 |
| | | ATGTTGGTTTCAAC | |
| 3872 | Forward | TGGTTTACACGG | 26 |
| | | TTGGGCTTTCAC | |
| 3873 | Forward | TGTCGAAGGTTTGA | 27 |
| | | AGAGATTGTCCAA | |
| 3874 | Forward | TGTCATTGCCC | 28 |
| | | ACGTCGATCA | |
| 3875 | Forward | TGCCTTGATTGACG | 29 |
| | | GTCATTGTGAAAT | |
| 3876 | Forward | TGACTACATCGA | 30 |
| | | CGAGTCCGAAGT | |
| 3877 | Forward | TTTTTTCCCTTCG | 31 |
| | | TCTGTGGTTGCCG | |
| 3878 | Forward | TCAAGGATGCTG | 32 |
| | | GCGTAATGGTCA | |
| 3879 | Forward | TTCCCTGGA | 33 |
| | | ACGGGACGC | |
| 4142 | Forward | TACAATGAGCTC | 34 |
| | | CGTGTCGCTCC | |
| 4143 | Forward | TCAGTCTAAGCGAGGT | 35 |
| | | ATTCTTACCTTGAAG | |
| 4144 | Forward | TACAGTCGAAAGAC | 36 |
| | | CGAAACGTCAATG | |
| 4145 | Forward | TACCAAGCCAAT | 37 |
| | | GACGAGTAGGG | |
| 4146 | Forward | TAGTCCAGTCTAA | 38 |
| | | TCTCCGTGCCAG | |
| 4147 | Forward | TCGATTCACTTG | 39 |
| | | CCTCTCAGCAG | |
| 4148 | Forward | TCGAGGCGG | 40 |
| | | TCCTCCTCA | |
| 4149 | Forward | TAGTCGAACTTC | 41 |
| | | AGGTCTGGCGA | |
| 4150 | Forward | TAGCAGCTCTAGCTCT | 42 |
| | | AGTACATATGCTAAC | |
| 4151 | Forward | TACCATTTATTCTAG | 43 |
| | | CAGCTCTAGCTCTAG | |
| 4158 | Forward | TCAATGGCCTA | 44 |
| | | ACGGCTGAAC | |
| 4159 | Forward | TGGCCTAACG | 45 |
| | | GCTGAACCA | |
| 4160 | Forward | TGAACCAGCAACTT | 46 |
| | | GGAAGAATGAGAAG | |
| 4161 | Forward | TGAACCAGCAACT | 47 |
| | | TGGAAGAATGAG | |
| 4312 | Forward | TGGATCTAGTAATCCA | 48 |
| | | CTAGGAATTACAGCAA | |
| 4313 | Forward | TGCAACATTAAACCG | 49 |
| | | ATTCTTCAGTCTTC | |
| 4314 | Forward | TTCTTCAGTCTTCACT | 50 |
| | | ACTTATTACCATTCA | |
| 4315 | Forward | TGTTGCAGTTAAA | 51 |
| | | ACGTCCGTAGTC | |
| 4316 | Forward | TGAGGTGGCCTG | 52 |
| | | GTCTTCATTGA | |
| 4317 | Forward | TGCAGGCTTTTAA | 53 |
| | | GCTTGAATGTGTT | |
| 4318 | Forward | TCGATTGAATGGTTA | 54 |
| | | TAGTGAGCATATGGG | |
| 4319 | Forward | TAATTTAGCGGA | 55 |
| | | TCGCATGGCCTT | |
| 4320 | Forward | TGACGTTTTCATTGATC | 56 |
| | | AAGGTCTAAAGTTAAG | |
| 4321 | Forward | TGGTTAATTCCTTTAA | 57 |
| | | TTAACGCAAACCCTT | |
| 4322 | Forward | TCATGCTAATCGTACGAC | 58 |
| | | ATCATATTAAAAATGATG | |
| 4323 | Forward | TGAACCAGCAACTTGGA | 59 |
| | | GGAATAAATGTAATGAT | |
| 4324 | Forward | TCATGGGCGTCT | 60 |
| | | ATCAGGATGGGA | |
| 4325 | Forward | TGGATCTTAAATTCA | 61 |
| | | AAGGTGCAGAGAAGG | |
| 4326 | Forward | TGTCTGAAGGAG | 62 |
| | | CGCAATGAGAAG | |
| 4327 | Forward | TCGATGGTTCTT | 63 |
| | | CAACGCTTTCG | |
| 4328 | Forward | TTTCTGCGAAAATT | 64 |
| | | GTTTTGGCAAATTG | |
| 4329 | Forward | TGCGAAAATTGTTTTGG | 65 |
| | | CAAATTGTTTATTCCTC | |
| 4330 | Forward | TGCCAGGGATGCC | 66 |
| | | CTGTTTCTTTTTG | |
| 4331 | Forward | TCGCAGGTTGG | 67 |
| | | TTGCGCTTTG | |
| 4332 | Forward | TCGCATGAGCGTC | 68 |
| | | TACTTCAACGAG | |
| 4333 | Forward | TCAACTGTTACATCAA | 69 |
| | | CAATCCAGCTTTTAC | |
| 4334 | Forward | TAAGCGCAAGG | 70 |
| | | GTCATGCTCTG | |
| 3029 | Reverse | TCCACGTTCAATT | 71 |
| | | AAGCAACAAGGAC | |
| 3030 | Reverse | TTCTCACCCTCTGT | 72 |
| | | GACGGCCTGTTCC | |
| 3031 | Reverse | TCAGCTATGCTCTT | 73 |
| | | ACTCAAATCCATC | |
| 3032 | Reverse | TGACAATGTCTT | 74 |
| | | CAACCCGGATC | |
| 3766 | Reverse | TCTGACAATGTCT | 75 |
| | | TCAACCCGGATC | |
| 3815 | Reverse | TGGCATGCCC | 76 |
| | | TCCGGAATAC | |
| 3816 | Reverse | TGTGCGTTCAAAGA | 77 |
| | | TTCGATGATTCAC | |
| 3856 | Reverse | TGCCGACTTCCCTTATCT | 78 |
| | | ACATTATTCTATCAACT | |
| 3857 | Reverse | TCCCAACAGCTATGC | 79 |
| | | TCTTACTCAAATCC | |
| 3858 | Reverse | TCCCAACAGCTACGC | 80 |
| | | TCTTACTCAAATCC | |
| 3859 | Reverse | TGGAGTTGAAAGTG | 81 |
| | | GTTTGGTCAATACC | |
| 3860 | Reverse | TCCTTTCGGCCAT | 82 |
| | | TGACCAATATTCC | |
| 3861 | Reverse | TGACATTCATCATTT | 83 |
| | | TCTGCTTGGACTACT | |
| 3862 | Reverse | TCCCCTTACTTTAAG | 84 |
| | | GTAGCCAAATTATC | |
| 3913 | Reverse | TGCCATATTACTCT | 85 |
| | | TGAGGTGGAATGC | |
| 3863 | Reverse | TGCCCGAACTGTATTT | 86 |
| | | CAACTTATAGCATATC | |
| 3864 | Reverse | TCTGACGACAACA | 87 |
| | | ATGTAACGCCTG | |
| 3865 | Reverse | TCTGACGACAACA | 88 |
| | | ATGTAACGCCTG | |
| 3866 | Reverse | TCGGACGTACT | 89 |
| | | AGTCTGGCGGT | |
| 3867 | Reverse | TCATTATTGCTAACG | 90 |
| | | TACTCTTCAGGTGG | |
| 3868 | Reverse | TGTCTGTAACCGTCTA | 91 |
| | | TTGTTTTGAGTTTCA | |
| 3869 | Reverse | TTTGGTGGATCGT | 92 |
| | | TCTTGGAGTCACC | |
| 3870 | Reverse | TCAGCTGGGGCGAAA | 93 |
| | | GTGACAACCATACC | |
| 3871 | Reverse | TGTGTGACAATCCAA | 94 |
| | | GACTGGAGAGTAACC | |
| 3912 | Reverse | TTTCGTGGATCGT | 95 |
| | | TCTTGGAGTCACC | |
| 3872 | Reverse | TGTTAGTCCATTTC | 96 |
| | | TTGGTCTTTGGGT | |
| 3873 | Reverse | TGCAAACAAATTTCC | 97 |
| | | AAGTGCAATTCACC | |
| 3874 | Reverse | TACCTCTTTCTTGTTCA | 98 |
| | | TCTTTTCTGGTATCCAT | |
| 3875 | Reverse | TGCATGGTTTTGGTTAT | 99 |
| | | CAGATTTACCATCACC | |
| 3876 | Reverse | TAGGTTGCGGC | 100 |
| | | AGCCACAGAC | |
| 3877 | Reverse | TGGTGCCGG | 101 |
| | | CCTCACCCT | |
| 3878 | Reverse | TCGAAGCTCT | 102 |
| | | CACCTGCGTT | |
| 3879 | Reverse | TGCATTCCCAAAC | 103 |
| | | TACTCGACTCGT | |
| 4142 | Reverse | TGTCATCTTCTCT | 104 |
| | | CGGTTCTGCTTGG | |
| 4143 | Reverse | TCAGGAGCGAC | 105 |
| | | ACGGAGCTCA | |
| 4144 | Reverse | TACGCGGGCTAT | 106 |
| | | CGGATTAAACG | |
| 4145 | Reverse | TGCTGCACATAAT | 107 |
| | | CTATGCTCTGGAC | |
| 4146 | Reverse | TACACCTATTCAAA | 108 |
| | | ACTCCGATGGGTTG | |
| 4147 | Reverse | TCACTCTCCCTAC | 109 |
| | | TCGTCATTGGCT | |
| 4148 | Reverse | TCCAGGAGGTA | 110 |
| | | AGGTCCAGCA | |
| 4149 | Reverse | TGGACCACCAG | 111 |
| | | GAGGTAAGGTC | |
| 4150 | Reverse | TAAAGATAAAGTATG | 112 |
| | | GATGCATTGGTGCC | |
| 4151 | Reverse | TGCCTTTTCAGCAT | 113 |
| | | TTGAGCTAATACC | |
| 4158 | Reverse | TCCCTAGAATTTCATA | 114 |
| | | CGATATATTCGTTTCG | |
| 4159 | Reverse | TGCTGGCACGTAA | 115 |
| | | TTTGGTCAAGAC | |
| 4160 | Reverse | TGCTGGCACGTAA | 116 |
| | | TTTGGTCAAGAC | |
| 4161 | Reverse | TCCCTAGAATTTCATA | 117 |
| | | CGATATATTCGTTTCG | |
| 4312 | Reverse | TTGGGTGCATAGAATAAG | 118 |
| | | AATAAAGCTAATACTAAG | |
| 4313 | Reverse | TAAACCCTATCAGCAT | 119 |
| | | TTGCTGTAATTCCTA | |
| 4314 | Reverse | TAAACCCTATCAGCAT | 120 |
| | | TTGCTGTAATTCCTA | |
| 4315 | Reverse | TGCGAGCTTGATC | 121 |
| | | AATGAAGACCAGG | |
| 4316 | Reverse | TGCTTTAAACACTCTG | 122 |
| | | ATTTGCTCATGGTAAT | |
| 4317 | Reverse | TTTCACCTCTAGCG | 123 |
| | | ACCAAATACAAATG | |
| 4318 | Reverse | TCTTCCTCTAAATAG | 124 |
| | | CCTAGTTTGCCATAG | |
| 4319 | Reverse | TCATGATAGGGCAG | 125 |
| | | AAAATCGAGTGGA | |
| 4320 | Reverse | TCAATCTCTAGTCGGC | 126 |
| | | ATAGTTTGTGGTTAAG | |
| 4321 | Reverse | TCCCTATTCTCTAAC | 127 |
| | | CATTTTTTTAGCGA | |
| 4322 | Reverse | TGGCATCTTAAAGC | 128 |
| | | GAAACCTTAGTTTC | |
| 4323 | Reverse | TGCTGGCACGTAA | 129 |
| | | TTTGGTCAAGAC | |
| 4324 | Reverse | TCCCTTCTCTGCACCT | 130 |
| | | TTAAATTTAAGATCC | |
| 4325 | Reverse | TCTTCTCATTGCG | 131 |
| | | CTCCTTCAGACA | |
| 4326 | Reverse | TCGCCCGAAAG | 132 |
| | | CGTTGAAGAAC | |
| 4327 | Reverse | TGATCGAACGCT | 133 |
| | | TTCCAGAGATGG | |
| 4328 | Reverse | TCCCTCGAGATAT | 134 |
| | | TCAGTGCTATACC | |
| 4329 | Reverse | TCCCTGAGATAAATATT | 135 |
| | | TTCATACTCCCTCGAG | |
| 4330 | Reverse | TACCGAATACAGG | 136 |
| | | GTTCATGCCGAG | |
| 4331 | Reverse | TCATACCTCGCACC | 137 |
| | | TCTTCAAAATCTG | |
| 4332 | Reverse | TACCGGGCTCA | 138 |
| | | AGATCGACGAG | |
| 4333 | Reverse | TCAGTTTGCATTT | 139 |
| | | GCTACCAAACTGG | |
| 4334 | Reverse | TCTCCTTTGTTTGTG | 140 |
| | | AAGTAAAAGCTGGA | |
| 4832 | Forward | TCCGCTTTCAAA | 141 |
| | | GAGTCAGGTTGT | |
| 4832 | Reverse | TGGTACTTGTTCG | 142 |
| | | CTATCGGTCTCTC | |
| 4833 | Forward | TCTTGGATTGACCG | 143 |
| | | AAGACAAACTACTG | |
| 4833 | Reverse | TCGGTATCTAATC | 144 |
| | | GTCTTCGATCCCT | |
| 4834 | Forward | TCGAAGACGATTAGAT | 145 |
| | | ACCGTCGTAGTCTTA | |
| 4834 | Reverse | TCCAGAACCCAAAAAC | 146 |
| | | TTTACTTTCGCTAAG | |
| 4835 | Forward | TCCAGCGGATTAC | 147 |
| | | CATGAGCAAATC | |
| 4835 | Reverse | TCCTAGAGTCGTATTTC | 148 |
| | | ATTATTCCATGCTAAC | |
| 4836 | Forward | TCTTGGATTGACCG | 149 |
| | | AAGACAAACTACTG | |
| 4836 | Reverse | TCTCTAGTCGGCAT | 150 |
| | | CGTTTGTGGTTAAG | |
| 4837 | Forward | TGCCGCGGTGC | 151 |
| | | TCACTCTTTC | |
| 4837 | Reverse | TGTATCGGCCG | 152 |
| | | TTGCGGAAGTC | |

TABLE 1B
Primer Pair Names, Amplicon Coordinates and Reference Amplicon
Lengths

| Prim- | | Ref- |
|---|---|---|
| er | | erence |
| Pair | | Am- |
| Num- | | plicon |
| ber | Primer Pair Name/Hybridization Coordinates | length |
| 3029 | 25SCANDIDA_X70659_996_1129 | 134 |
| 3030 | 25SFUNG_X70659_134_261 | 128 |
| 3031 | 25SFUNG_X70659_697_834 | 138 |
| 3032 | 25SFUNG_X70659_2472_2615 | 144 |
| 3766 | 25SFUNG_X70659_2470_2617 | 148 |
| 3815 | 5P8SRNA_AY342214-165-322_34_143 | 110 |
| 3816 | 5P8SRNA_AY342214-165-322_34_111 | 78 |
| 3856 | 25SCANDIDA_X70659_1826_1946 | 121 |
| 3857 | 25SCANDIDA_X70659_733_839 | 107 |
| 3858 | 25SCANDIDA_X70659_727_839 | 113 |
| 3859 | ACT1_X16377-1691-2809_754_838 | 85 |
| 3860 | ASP-MITSSURRNA_NC007445-8943-10314_241_321 | 81 |
| 3861 | ASP-MITSSURRNA_NC007445-8943-10314_630_724 | 95 |
| 3862 | ASP-MITSSURRNA_NC007445-8943-10314_13_120 | 108 |
| 3863 | CAN-MITSSURRNA_AF285261-27022-28483_1290_1413 | 124 |
| 3864 | CAN-MITSSURRNA_AF285261-27022-28483_809_958 | 150 |
| 3865 | CAN-MITSSURRNA_AF285261-27022-28483_844_958 | 115 |
| 3866 | CAN-MITSSURRNA_AF285261-27022-28483_664_808 | 145 |
| 3867 | CANGL-MITSSURRNA_NC004691-18404-20053_836_954 | 119 |
| 3868 | CANGL-MITSSURRNA_NC004691-18404-20053_836_982 | 147 |
| 3869 | EFT1A_NW139597-1412-2152_423_521 | 99 |
| 3870 | EFT1A_NW139597-1412-2152_260_368 | 109 |
| 3871 | EFT1A_NW139597-1412-2152_477_612 | 136 |
| 3872 | EFT2_AF107286-3-2441_591_730 | 140 |
| 3873 | EFT2_AF107286-3-2441_1464_1580 | 117 |
| 3874 | EFT2_AF107286-3-2441_21_154 | 134 |
| 3875 | GSC1_D88815-723-6308_3141_3261 | 121 |
| 3876 | PYROA-ASP_CM000173-2050630-2050004_21_123 | 103 |
| 3877 | PYROA-ASP_CM000173-2050630-2050004_91_190 | 100 |
| 3878 | 25SFUSARIUM_DQ286135_559_688 | 130 |
| 3879 | 25SFUSARIUM_DQ286135_148_246 | 99 |
| 3912 | EFT1A_NW139597-1412-2152_423_521_2 | 99 |
| 3913 | ASP-MITSSURRNA_NC007445-8943-10314_680_791 | 112 |
| 4142 | ACT1_XM772884-13-1131_265_354 | 90 |
| 4143 | ACT1_XM772884-13-1131_168_290 | 123 |
| 4144 | CRYPTO-MITSSURNA_NC004336-10422-11799_787_872 | 86 |
| 4145 | CRYPTO-MITSSURNA_NC004336-10422-11799_234_320 | 87 |
| 4146 | CRYPTO-MITSSURNA_NC004336-10422-11799_441_517 | 77 |
| 4147 | CRYPTO-MITSSURNA_NC004336-10422-11799_190_263 | 74 |
| 4148 | 18SCRYPTO_AE017342-289742-287938_649_702 | 54 |
| 4149 | 18SCRYPTO_AE017342-289742-287938_630_708 | 79 |
| 4150 | CRYPTO-MITCYTB_AB105930_1268_1381 | 114 |
| 4151 | CRYPTO-MITCYTB_AB105930_1256_1356 | 101 |
| 4158 | COCCID-MIT_AASO01004342-13441-12000_326_419 | 94 |
| 4159 | COCCID-MIT_AASO01004342-13441-12000_330_476 | 147 |
| 4160 | COCCID-MIT_AASO01004342-13441-12000_342_476 | 135 |
| 4161 | COCCID-MIT_AASO01004342-13441-12000_342_419 | 78 |
| 4312 | MUCOR-MIT-CYTB_AB025730_169_303 | 135 |
| 4313 | MUCOR-MIT-CYTB_AB025730_76_216 | 141 |
| 4314 | MUCOR-MIT-CYTB_AB025730_92_216 | 125 |
| 4315 | MUCOR-18SRNA_AF113430_577_652 | 76 |
| 4316 | MUCOR-18SRNA_AF113430_619_751 | 133 |
| 4317 | MUCOR-18SRNA_AF113430_748_884 | 137 |
| 4318 | MUCOR-18SRNA_AF113430_1646_1747 | 102 |
| 4319 | MUCOR-18SRNA_AF113430_223_286 | 64 |
| 4320 | MUCOR-18SRNA_AF113430_931_1025 | 95 |
| 4321 | AJEL-MIT_AAJI01002868-17184-16232_1053_1137 | 85 |
| 4322 | AJEL-MIT_AAJI01002868-17184-16232_47_180 | 134 |
| 4323 | AJEL-MIT_AAJI01002868-17184-16232_341_457 | 117 |
| 4324 | AJEL-CHS2_M82943_170_300 | 131 |
| 4325 | AJEL-CHS2_M82943_269_349 | 81 |
| 4326 | AJEL-CHS2_M82943_325_390 | 66 |
| 4327 | AJEL-CHS2_M82943_363_479 | 117 |
| 4328 | PNEUMO-MITLSURNA_EF439817_32_107 | 76 |
| 4329 | PNEUMO-MITLSURNA_EF439817_36_131 | 96 |
| 4330 | PNEUMO-MSGP_AY845945_874_982 | 109 |
| 4331 | PNEUMO-MSGP_AY845945_1237_1312 | 76 |
| 4332 | FUS-BTUBULIN_DQ220228_75_198 | 124 |
| 4333 | FUS-TOPOII_EU214571_210_302 | 93 |
| 4334 | FUS-TOPOII_EU214571_151_257 | 107 |
| 4832 | 26SMUCOR_M26190_411_514 | 104 |
| 4833 | 18SMUCOR_AF113430_886_987 | 102 |
| 4834 | 18SMUCOR_AF113430_967_1083 | 117 |
| 4835 | 18SMUCOR_AF113430_712_803 | 92 |
| 4836 | 18SMUCOR_AF113430_886_1021 | 136 |
| 4837 | SCEDBTUB_AM261879_78_186 | 109 |

TABLE 1C
Individual Primer Names and Hybridization Coordinates

| Primer | | |
|---|---|---|
| Pair | Primer | Individual Primer Name/ |
| Number | Direction | Hybridization Coordinates |
| 3029 | Forward | 25SCANDIDA_X70659_996_1022_F |
| 3029 | Reverse | 25SCANDIDA_X70659_1104_1129_R |
| 3030 | Forward | 25SFUNG_X70659_134_158_F |
| 3030 | Reverse | 25SFUNG_X70659_235_261_R |
| 3031 | Forward | 25SFUNG_X70659_697_722_F |
| 3031 | Reverse | 25SFUNG_X70659_808_834_R |
| 3032 | Forward | 25SFUNG_X70659_2472_2496_F |
| 3032 | Reverse | 25SFUNG_X70659_2593_2615_R |
| 3766 | Forward | 25SFUNG_X70659_2470_2496_F |
| 3766 | Reverse | 25SFUNG_X70659_2593_2617_R |
| 3815 | Forward | 5P8SRNA_AY342214-165-322_34_57_F |
| 3815 | Reverse | 5P8SRNA_AY342214-165-322_124_143_R |
| 3816 | Forward | 5P8SRNA_AY342214-165-322_34_57_F |
| 3816 | Reverse | 5P8SRNA_AY342214-165-322_85_111_R |
| 3856 | Forward | 25SCANDIDA_X70659_1826_1855_F |
| 3856 | Reverse | 25SCANDIDA_X70659_1912_1946_R |
| 3857 | Forward | 25SCANDIDA_X70659_733_758_F |
| 3857 | Reverse | 25SCANDIDA_X70659_811_839_R |
| 3858 | Forward | 25SCANDIDA_X70659_727_756_F |
| 3858 | Reverse | 25SCANDIDA_X70659_811_839_2_R |
| 3859 | Forward | ACT1_X16377-1691-2809_754_780_F |
| 3859 | Reverse | ACT1_X16377-1691-2809_811_838_R |
| 3860 | Forward | ASP-MITSSURRNA_NC007445-8943- |
| | | 10314_241_263_F |
| 3860 | Reverse | ASP-MITSSURRNA_NC007445-8943- |
| | | 10314_296_321_R |
| 3861 | Forward | ASP-MITSSURRNA_NC007445-8943- |
| | | 10314_630_657_F |
| 3861 | Reverse | ASP-MITSSURRNA_NC007445-8943- |
| | | 10314_695_724_R |
| 3862 | Forward | ASP-MITSSURRNA_NC007445-8943- |
| | | 10314_13_38_F |
| 3862 | Reverse | ASP-MITSSURRNA_NC007445-8943- |
| | | 10314_92_120_R |
| 3863 | Forward | CAN-MITSSURRNA_AF285261-27022- |
| | | 28483_1290_1325_F |
| 3863 | Reverse | CAN-MITSSURRNA_AF285261-27022- |
| | | 28483_1382_1413_R |
| 3864 | Forward | CAN-MITSSURRNA_AF285261-27022- |
| | | 28483_809_838_F |
| 3864 | Reverse | CAN-MITSSURRNA_AF285261-27022- |
| | | 28483_934_958_R |
| 3865 | Forward | CAN-MITSSURRNA_AF285261-27022- |
| | | 28483_844_873_F |
| 3865 | Reverse | CAN-MITSSURRNA_AF285261-27022- |
| | | 28483_934_958_R |
| 3866 | Forward | CAN-MITSSURRNA_AF285261-27022- |
| | | 28483_664_689_F |
| 3866 | Reverse | CAN-MITSSURRNA_AF285261-27022- |
| | | 28483_787_808_R |
| 3867 | Forward | CANGL-MITSSURRNA_NC004691-18404- |
| | | 20053_836_865_F |
| 3867 | Reverse | CANGL-MITSSURRNA_NC004691-18404- |
| | | 20053_926_954_R |
| 3868 | Forward | CANGL-MITSSURRNA_NC004691-18404- |
| | | 20053_836_865_F |
| 3868 | Reverse | CANGL-MITSSURRNA_NC004691-18404- |
| | | 20053_952_982_R |
| 3869 | Forward | EFT1A_NW139597-1412-2152_423_450_F |
| 3869 | Reverse | EFT1A_NW139597-1412-2152_496_521_R |
| 3870 | Forward | EFT1A_NW139597-1412-2152_260_293_F |
| 3870 | Reverse | EFT1A_NW139597-1412-2152_340_368_R |
| 3871 | Forward | EFT1A_NW139597-1412-2152_477_507_F |
| 3871 | Reverse | EFT1A_NW139597-1412-2152_583_612_R |
| 3872 | Forward | EFT2_AF107286-3-2441_591_614_F |
| 3872 | Reverse | EFT2_AF107286-3-2441_704_730_R |
| 3873 | Forward | EFT2_AF107286-3-2441_1464_1490_F |
| 3873 | Reverse | EFT2_AF107286-3-2441_1552_1580_R |
| 3874 | Forward | EFT2_AF107286-3-2441_21_41_F |
| 3874 | Reverse | EFT2_AF107286-3-2441_121_154_R |
| 3875 | Forward | GSC1_D88815-723-6308_3141_3167_F |
| 3875 | Reverse | GSC1_D88815-723-6308_3229_3261_R |
| 3876 | Forward | PYROA-ASP_CM000173-2050630- |
| | | 2050004_21_44_F |
| 3876 | Reverse | PYROA-ASP_CM000173-2050630- |
| | | 2050004_103_123_R |
| 3877 | Forward | PYROA-ASP_CM000173-2050630- |
| | | 2050004_91_116_F |
| 3877 | Reverse | PYROA-ASP_CM000173-2050630- |
| | | 2050004_173_190_R |
| 3878 | Forward | 25SFUSARIUM_DQ286135_559_582_F |
| 3878 | Reverse | 25SFUSARIUM_DQ286135_669_688_R |
| 3879 | Forward | 25SFUSARIUM_DQ286135_148_165_F |
| 3879 | Reverse | 25SFUSARIUM_DQ286135_222_246_R |
| 3912 | Forward | EFT1A_NW139597-1412-2152_423_450_F |
| 3912 | Reverse | EFT1A_NW139597-1412-2152_496_521_2_R |
| 3913 | Forward | ASP-MITSSURRNA_NC007445-8943- |
| | | 10314_680_708_F |
| 3913 | Reverse | ASP-MITSSURRNA_NC007445-8943- |
| | | 10314_765_791_R |
| 4142 | Forward | ACT1_XM772884-13-1131_265_287_F |
| 4142 | Reverse | ACT1_XM772884-13-1131_329_354_R |
| 4143 | Forward | ACT1_XM772884-13-1131_168_198_F |
| 4143 | Reverse | ACT1_XM772884-13-1131_270_290_R |
| 4144 | Forward | CRYP-MITSSURNA_NC004336-10422- |
| | | 11799_787_813_F |
| 4144 | Reverse | CRYP-MITSSURNA_NC004336-10422- |
| | | 11799_850_872_R |
| 4145 | Forward | CRYP-MITSSURNA_NC004336-10422- |
| | | 11799_234_256_F |
| 4145 | Reverse | CRYP-MITSSURNA_NC004336-10422- |
| | | 11799_295_320_R |
| 4146 | Forward | CRYP-MITSSURNA_NC004336-10422- |
| | | 11799_441_465_F |
| 4146 | Reverse | CRYP-MITSSURNA_NC004336-10422- |
| | | 11799_490_517_R |
| 4147 | Forward | CRYP-MITSSURNA_NC004336-10422- |
| | | 11799_190_212_F |
| 4147 | Reverse | CRYP-MITSSURNA_NC004336-10422- |
| | | 11799_239_263_R |
| 4148 | Forward | 18SCRYPTO_AE017342-289742- |
| | | 287938_649_666_F |
| 4148 | Reverse | 18SCRYPTO_AE017342-289742- |
| | | 287938_682_702_R |
| 4149 | Forward | 18SCRYPTO_AE017342-289742- |
| | | 287938_630_652_F |
| 4149 | Reverse | 18SCRYPTO_AE017342-289742- |
| | | 287938_687_708_R |
| 4150 | Forward | CRYPTO-MITCYTB_AB105930_1268_1298_F |
| 4150 | Reverse | CRYPTO-MITCYTB_AB105930_1353_1381_R |
| 4151 | Forward | CRYPTO-MITCYTB_AB105930_1256_1285_F |
| 4151 | Reverse | CRYPTO-MITCYTB_AB105930_1330_1356_R |
| 4158 | Forward | COCCID-MIT_AAS001004342-13441- |
| | | 12000_326_346_F |
| 4158 | Reverse | COCCID-MIT_AAS001004342-13441- |
| | | 12000_388_419_R |
| 4159 | Forward | COCCID-MIT_AAS001004342-13441- |
| | | 12000_330_348_F |
| 4159 | Reverse | COCCID-MIT_AAS001004342-13441- |
| | | 12000_452_476_R |
| 4160 | Forward | COCCID-MIT_AAS001004342-13441- |
| | | 12000_342_369_F |
| 4160 | Reverse | COCCID-MIT_AAS001004342-13441- |
| | | 12000_452_476_R |
| 4161 | Forward | COCCID-MIT_AAS001004342-13441- |
| | | 12000_342_366_F |
| 4161 | Reverse | COCCID-MIT_AAS001004342-13441- |
| | | 12000_388_419_R |
| 4312 | Forward | MUCOR-MIT-CYTB_AB025730_169_200_F |
| 4312 | Reverse | MUCOR-MIT-CYTB_AB025730_268_303_R |
| 4313 | Forward | MUCOR-MIT-CYTB_AB025730_76_104_F |
| 4313 | Reverse | MUCOR-MIT-CYTB_AB025730_186_216_R |
| 4314 | Forward | MUCOR-MIT-CYTB_AB025730_92_122_F |
| 4314 | Reverse | MUCOR-MIT-CYTB_AB025730_186_216_R |
| 4315 | Forward | MUCOR-18SRNA_AF113430_577_601_F |
| 4315 | Reverse | MUCOR-18SRNA_AF113430_627_652_R |
| 4316 | Forward | MUCOR-18SRNA_AF113430_619_641_F |
| 4316 | Reverse | MUCOR-18SRNA_AF113430_720_751_R |
| 4317 | Forward | MUCOR-18SRNA_AF113430_748_773_F |
| 4317 | Reverse | MUCOR-18SRNA_AF113430_857_884_R |
| 4318 | Forward | MUCOR-18SRNA_AF113430_1646_1675_F |
| 4318 | Reverse | MUCOR-18SRNA_AF113430_1718_1747_R |
| 4319 | Forward | MUCOR-18SRNA_AF113430_223_246_F |
| 4319 | Reverse | MUCOR-18SRNA_AF113430_260_286_R |
| 4320 | Forward | MUCOR-18SRNA_AF113430_931_963_F |
| 4320 | Reverse | MUCOR-18SRNA_AF113430_994_1025_R |
| 4321 | Forward | AJEL-MIT_AAJI01002868-17184- |
| | | 16232_1053_1083_F |
| 4321 | Reverse | AJEL-MIT_AAJI01002868-17184- |
| | | 16232_1109_1137_R |
| 4322 | Forward | AJEL-MIT_AAJI01002868-17184- |
| | | 16232_47_82_F |
| 4322 | Reverse | AJEL-MIT_AAJI01002868-17184- |
| | | 16232_153_180_R |
| 4323 | Forward | AJEL-MIT_AAJI01002868-17184- |
| | | 16232_341_374_F |
| 4323 | Reverse | AJEL-MIT_AAJI01002868-17184- |
| | | 16232_433_457_R |
| 4324 | Forward | AJEL-CHS2_M82943_170_193_F |
| 4324 | Reverse | AJEL-CHS2_M82943_270_300_R |
| 4325 | Forward | AJEL-CHS2_M82943_269_298_F |
| 4325 | Reverse | AJEL-CHS2_M82943_325_349_R |
| 4326 | Forward | AJEL-CHS2_M82943_325_348_F |
| 4326 | Reverse | AJEL-CHS2_M82943_369_390_R |
| 4327 | Forward | AJEL-CHS2_M82943_363_385_F |
| 4327 | Reverse | AJEL-CHS2_M82943_456_479_R |
| 4328 | Forward | PNEUMO-MITLSURNA_EF439817_32_59_F |
| 4328 | Reverse | PNEUMO-MITLSURNA_EF439817_82_107_R |
| 4329 | Forward | PNEUMO-MITLSURNA_EF439817_36_69_F |
| 4329 | Reverse | PNEUMO-MITLSURNA_EF439817_99_131_R |
| 4330 | Forward | PNEUMO-MSGP_AY845945_874_899_F |
| 4330 | Reverse | PNEUMO-MSGP_AY845945_958_982_R |
| 4331 | Forward | PNEUMO-MSGP_AY845945_1237_1257_F |
| 4331 | Reverse | PNEUMO-MSGP_AY845945_1286_1312_R |
| 4332 | Forward | FUS-BTUBULIN_DQ220228_75_99_F |
| 4332 | Reverse | FUS-BTUBULIN_DQ220228_177_198_R |
| 4333 | Forward | FUS-TOPOII_EU214571_210_240_F |
| 4333 | Reverse | FUS-TOPOII_EU214571_277_302_R |
| 4334 | Forward | FUS-TOPOII_EU214571_151_172_F |
| 4334 | Reverse | FUS-TOPOII_EU214571_229_257_R |
| 4832 | Forward | 26SMUCOR_M26190_411_434_F |
| 4832 | Reverse | 26SMUCOR_M26190_489_514_R |
| 4833 | Forward | 18SMUCOR_AF113430_886_913_F |
| 4833 | Reverse | 18SMUCOR_AF113430_962_987_R |
| 4834 | Forward | 18SMUCOR_AF113430_967_997_F |
| 4834 | Reverse | 18SMUCOR_AF113430_1053_1083_R |
| 4835 | Forward | 18SMUCOR_AF113430_712_736_F |
| 4835 | Reverse | 18SMUCOR_AF113430_771_803_R |
| 4836 | Forward | 18SMUCOR_AF113430_886_913_F |
| 4836 | Reverse | 18SMUCOR_AF113430_994_1021_R |
| 4837 | Forward | SCEDBTUB_AM261879_78_98_F |
| 4837 | Reverse | SCEDBTUB_AM261879_165_186_R |

It is noted that the primer pairs in Table 1A could be combined into a single panel for detection one or more fungi (e.g., clinically relevant fungi). The primers and primer pairs of Table 1 could be used, for example, to detect human and animal infections. These primers and primer pairs may also be grouped (e.g., in panels or kits) for multiplex detection of other bioagents (e.g., in an assay for detection of blood borne infections). In particular embodiments, the primers are used in assays for testing product safety.

TABLE 2

| | | | | GenBank | |
|---|---|---|---|---|---|
| | | | | reference | GenBank |
| | | | | sequence | reference |
| pp num | target | Target groups | Description | accession | sequence gi |
| 3029 | 25S rRNA | Candida spp. (not | Primers to detect 25S of Candida | X70659.1 | 671812 |
| | | specific) | | | |
| 3030 | 25S rRNA | Candida + Aspergillus | Primers for detection of Candida and Aspergillus | X70659.1 | 671812 |
| | | (not specific) | | | |
| 3031 | 25S rRNA | Candida + Aspergillus | Primers for detection of Candida and Aspergillus | X70659.1 | 671812 |
| | | (not specific) | | | |
| 3032 | 25S rRNA | Candida + Aspergillus | Primers for detection of Candida and Aspergillus | X70659.1 | 671812 |
| | | (not specific) | | | |
| 3766 | 25S rRNA | Candida + Aspergillus | Fungal primer pair to replace primer pair 3032 in order to | X70659.1 | 671812 |
| | | (not specific) | separate forward and reverse strand masses for | | |
| | | | Aspergillus fumigatus. | | |
| 3815 | 5.8S rRNA | All | Broad-spectrum fungal primers directed at the 5.8S rRNA | AY342214.1 | 33469617 |
| 3816 | 5.8S rRNA | All | Broad-spectrum fungal primers directed at the 5.8S rRNA | AY342214.1 | 33469617 |
| 3856 | 25S rRNA | Candida spp. (not | Primers targeted to the Candida 25S rRNA gene for | X70659.1 | 671812 |
| | | specific) | detection and differentiation of the major Candida species | | |
| | | | (albicans, tropicalis, glabrata) | | |
| 3857 | 25S rRNA | Candida spp. (not | Primers targeted to the Candida 25S rRNA gene for | X70659.1 | 671812 |
| | | specific) | detection and differentiation of the major Candida species | | |
| | | | (albicans, tropicalis, glabrata) | | |
| 3858 | 25S rRNA | Candida spp. (not | Primers targeted to the Candida 25S rRNA gene for | X70659.1 | 671812 |
| | | specific) | detection and differentiation of the major Candida species | | |
| | | | (albicans, tropicalis, glabrata) | | |
| 3859 | Actin | Candida spp. | Primers targeted to Candida spp. actin gene ACT1 | X16377.1 | 2492 |
| 3860 | mito-ss rRNA | Aspergillus spp. | Primers targeted to the Aspregillus spp. mitochondrial | NC_007445.1 | 77019995 |
| | | | SSU rRNA gene. | | |
| 3861 | mito-ss rRNA | Aspergillus spp. | Primers targeted to the Aspregillus spp. mitochondrial | NC_007445.1 | 77019995 |
| | | | SSU rRNA gene. | | |
| 3862 | mito-ss rRNA | Aspergillus spp. | Primers targeted to the Aspregillus spp. mitochondrial | NC_007445.1 | 77019995 |
| | | | SSU rRNA gene. | | |
| 3913 | mito-ss rRNA | Aspergillus spp. | Primers targeted to the Aspergillus spp. mitochondrial | NC_007445.1 | 77019995 |
| | | | SSU rRNA gene to detect and differentiate Aspergillus | | |
| | | | spp. | | |
| 3863 | mito-ss rRNA | Candida albicans | Primers targeted to the mitochondrial SSU rRNA gene | AF285261.1 | 12539616 |
| | | | from Candida albicans group species. | | |
| 3864 | mito-ss rRNA | Candida albicans | Primers targeted to the mitochondrial SSU rRNA gene | AF285261.1 | 12539616 |
| | | | from Candida albicans group species. | | |
| 3865 | mito-ss rRNA | Candida albicans | Primers targeted to the mitochondrial SSU rRNA gene | AF285261.1 | 12539616 |
| | | | from Candida albicans group species. | | |
| 3866 | mito-ss rRNA | Candida albicans | Primers targeted to the mitochondrial SSU rRNA gene | AF285261.1 | 12539616 |
| | | | from Candida albicans group species. | | |
| 3867 | mito-ss rRNA | Candida glabrata | Primers targeted to the mitochondrial SSU rRNA gene | NC_004691.1 | 29570601 |
| | | | from Candida glabrata group species. | | |
| 3868 | mito-ss rRNA | Candida glabrata | Primers targeted to the mitochondrial SSU rRNA gene | NC_004691.1 | 29570601 |
| | | | from Candida glabrata group species. | | |
| 3869 | EFT-la | Candida spp. | Primers targeted to elongation factor 1 alpha for detection | NW_139597.1 | 68488547 |
| | | | and differentiation of Candida species | | |
| 3870 | EFT-1a | Candida spp. | Primers targeted to elongation factor 1 alpha for detection | NW_139597.1 | 68488547 |
| | | | and differentiation of Candida species | | |
| 3871 | EFT-1a | Candida spp. | Primers targeted to elongation factor 1 alpha for detection | NW_139597.1 | 68488547 |
| | | | and differentiation of Candida species | | |
| 3912 | EFT-1a | Candida spp. | Replacement for pp 3869 (Ef1-a) that had a problem with | NW_139597.1 | 68488547 |
| | | | the reverse primer hair-pinning | | |
| 3872 | EFT2 | Candida spp. | Primers targeted to EFT-2 for the detection and | AF107286.1 | 8927037 |
| | | | discrimination of Candida species | | |
| 3873 | EFT2 | Candida spp. | Primers targeted to EFT-2 for the detection and | AF107286.1 | 8927037 |
| | | | discrimination of Candida species | | |
| 3874 | EFT2 | Candida spp. | Primers targeted to EFT-2 for the detection and | AF107286.1 | 8927037 |
| | | | discrimination of Candida species | | |
| 3875 | GSC1 | Candida spp. | Primers targeted to the GSC1 gene (beta-1,3-glucan | D88815.1 | 2274846 |
| | | | synthase catalytic subunit 1) for detection and | | |
| | | | discrimination of Candida species. | | |
| 3876 | pyroA | Aspergillus spp. | Primers to pyroA exon 3 for detection and differentiation | CM000173.1 | 67471101 |
| | | | of Aspergillus species | | |
| 3877 | pyroA | Aspergillus spp. | Primers to pyroA exon 3 for detection and differentiation | CM000173.1 | 67471101 |
| | | | of Aspergillus species | | |
| 3878 | 25S rRNA | Fusarium spp. | Primers targeted to 25S rRNA for the detection of | DQ286135.1 | 82799387 |
| | | | Fusarium spp. | | |
| 3879 | 25S rRNA | Fusarium spp. | Primers targeted to 25S rRNA for the detection of | DQ286135.1 | 82799387 |
| | | | Fusarium spp. | | |
| 4142 | Actin | Cryptococcus | Primers targeting the actin gene from Cryptococcus | XM_772884.1 | 134106870 |
| | | neoformans | neoformans species | | |
| 4143 | Actin | Cryptococcus | Primers targeting the actin gene from Cryptococcus | XM_772884.1 | 134106870 |
| | | neoformans | neoformans species | | |
| 4144 | mito-ssu rRNA | Cryptococcus | Primers targeted to the mitochondrial ssu rRNA from | NC_004336.1 | 24080109 |
| | | neoformans | Cryptococcus neoformans | | |
| 4145 | mito-ssu rRNA | Cryptococcus | Primers targeted to the mitochondrial ssu rRNA from | NC_004336.1 | 24080109 |
| | | neoformans | Cryptococcus neoformans | | |
| 4146 | mito-ssu rRNA | Cryptococcus | Primers targeted to the mitochondrial ssu rRNA from | NC_004336.1 | 24080109 |
| | | neoformans | Cryptococcus neoformans | | |
| 4147 | mito-ssu rRNA | Cryptococcus | Primers targeted to the mitochondrial ssu rRNA from | NC_004336.1 | 24080109 |
| | | neoformans | Cryptococcus neoformans | | |
| 4148 | 18S (ssu) rRNA | Cryptococcus | Primers directed at the nuclear SSU rRNA from | AE017342.1 | 57223396 |
| | | neoformans | Cryptococcus neoformans | | |
| 4149 | 18S (ssu) rRNA | Cryptococcus | Primers directed at the nuclear SSU rRNA from | AE017342.1 | 57223396 |
| | | neoformans | Cryptococcus neoformans | | |
| 4150 | mito-cytochrome B | Cryptococcus | Primers targeted to the mitochondrial cytB gene from | AB105930.1 | 39725512 |
| | | neoformans | Cryptococcus neoformans | | |
| 4151 | mito-cytochrome B | Cryptococcus | Primers targeted to the mitochondrial cytB gene from | AB105930.1 | 39725512 |
| | | neoformans | Cryptococcus neoformans | | |
| 4158 | mito-ssu rRNA | Coccidioides immitis/ | Primers targeted to the Coccidioides immitis/posadasii | AASO01004342.1 | 115299853 |
| | | posadasii | mitochondrial 12S rRNA for differentiating | | |
| | | | C. posodasii and C. immitis | | |
| 4159 | mito-ssu rRNA | Coccidioides immitis/ | Primers targeted to the Coccidioides immitis/posadasii | AASO01004342.1 | 115299853 |
| | | posadasii | mitochondrial 12S rRNA for differentiating | | |
| | | | C. posodasii and C. immitis | | |
| 4160 | mito-ssu rRNA | Coccidioides immitis/ | Primers targeted to the Coccidioides immitis/posadasii | AASO01004342.1 | 115299853 |
| | | posadasii | Coccidioides immitis/mitochondrial 12S rRNA for | | |
| | | | differentiating C. posodasii and C. immitis | | |
| 4161 | mito-ssu rRNA | Coccidioides immitis/ | Primers targeted to the Coccidioides immitis/posadasii | AASO01004342.1 | 115299853 |
| | | posadasii | mitochondrial 12S rRNA for differentiating C. posodasii | | |
| | | | and C. immitis | | |
| 4312 | mito-cytochrome B | Mucor/Rhizopus | Primers targeted to the mitochondrial cytB gene from | AB025730.1 | 32170402 |
| | | | Mucor and Rhizopus species | | |
| 4313 | mito-cytochrome B | Mucor/Rhizopus | Primers targeted to the mitochondrial cytB gene from | AB025730.1 | 32170402 |
| | | | Mucor and Rhizopus species | | |
| 4314 | mito-cytochrome B | Mucor/Rhizopus | Primers targeted to the mitochondrial cytB gene from | AB025730.1 | 32170402 |
| | | | Mucor and Rhizopus species | | |
| 4315 | 18S (ssu) rRNA | Mucoraceae | Primers targted to the 18S rRNA gene from Mucoraceae | AF113430.1 | 6561325 |
| 4316 | 18S (ssu) rRNA | Mucoraceae | Primers targted to the 18S rRNA gene from Mucoraceae | AF113430.1 | 6561325 |
| 4317 | 18S (ssu) rRNA | Mucoraceae | Primers targted to the 18S rRNA gene from Mucoraceae | AF113430.1 | 6561325 |
| 4318 | 18S (ssu) rRNA | Mucoraceae | Primers targted to the 18S rRNA gene from Mucoraceae | AF113430.1 | 6561325 |
| 4319 | 18S (ssu) rRNA | Mucoraceae | Primers targted to the 18S rRNA gene from Mucoraceae | AF113430.1 | 6561325 |
| 4320 | 18S (ssu) rRNA | Mucoraceae | Primers targted to the 18S rRNA gene from Mucoraceae | AF113430.1 | 6561325 |
| 4321 | mito-ssu rRNA | Ajellomyces spp. | Primers targeted to the mitochondrial 12S rRNA (SSU | AAJI01002868.1 | 75860892 |
| | | | rRNA) from Ajellomyces spp. | | |
| 4322 | mito-ssu rRNA | Ajellomyces spp. | Primers targeted to the mitochondrial 12S rRNA (SSU | AAJI01002868.1 | 75860892 |
| | | | rRNA) from Ajellomyces spp. | | |
| 4323 | mito-ssu rRNA | Ajellomyces spp. | Primers targeted to the mitochondrial 12S rRNA (SSU | AAJI01002868.1 | 75860892 |
| | | | rRNA) from Ajellomyces spp. | | |
| 4324 | chitin synthase | Ajellomyces spp. | Primers targeted to chitin synthetase 2 from Ajellomyces | M82943.1 | 166959 |
| | | | for separating Ajellomyces (Histoplasma) capsulatus from | | |
| | | | Ajellomyces (Blastomyces) dermatitidis | | |
| 4325 | chitin synthase | Ajellomyces spp. | Primers targeted to chitin synthetase 2 from Ajellomyces | M82943.1 | 166959 |
| | | | for separating Ajellomyces (Histoplasma) capsulatus from | | |
| | | | Ajellomyces (Blastomyces) dermatitidis | | |
| 4326 | chitin synthase | Ajellomyces spp. | Primers targeted to chitin synthetase 2 from Ajellomyces | M82943.1 | 166959 |
| | | | for separating Ajellomyces (Histoplasma) capsulatus from | | |
| | | | Ajellomyces (Blastomyces) dermatitidis | | |
| 4327 | chitin synthase | Ajellomyces spp. | Primers targeted to chitin synthetase 2 from Ajellomyces | M82943.1 | 166959 |
| | | | for separating Ajellomyces (Histoplasma) capsulatus from | | |
| | | | Ajellomyces (Blastomyces) dermatitidis | | |
| 4328 | mito-Isu rRNA | Pneumocystis jirovecii | Primers targeted to the mitochondrial LSU rRNA gene | EF439817.1 | 126742335 |
| | | | from Pneumocystis jirovecii | | |
| 4329 | mito-Isu rRNA | Pneumocystis jirovecii | Primers targeted to the mitochondrial LSU rRNA gene | EF439817.1 | 126742335 |
| | | | from Pneumocystis jirovecii | | |
| 4330 | major surface | Pneumocystis jirovecii | Primers targeted to the major surface glycoprotein gene | AY845945.1 | 61679089 |
| | glycoprotein | | from Pneumocystis jirovecii (human variety of | | |
| | | | Pneumocystis carinii) | | |
| 4331 | major surface | Pneumocystis jirovecii | Primers targeted to the major surface glycoprotein gene | AY845945.1 | 61679089 |
| | glycoprotein | | from Pneumocystis jirovecii (human variety of | | |
| | | | Pneumocystis carinii) | | |
| 4332 | beta-tubulin | Fusarium spp. + semi- | Primers targeted to the beta-tubulin gene from Fusarium | DQ220228.1 | 77738420 |
| | | broad detection with | spp. | | |
| | | resolution of Fusarium | | | |
| | | spp. | | | |
| 4333 | Topoisomerase II | Fusarium solani/ | Primers targeted to Fusarium solani and oxysporum | EU214571.1 | 162532533 |
| | | oxysporum | topoisomerase II | | |
| 4334 | Topoisomerase II | Fusarium solani/ | Primers targeted to Fusarium solani and oxysporum | EU214571.1 | 162532533 |
| | | oxysporum | topoisomerase II | | |

TABLE 2B

| Primer | | | GenBank | |
|---|---|---|---|---|
| Pair | | | Accession | GenBank |
| Number | Target | Target Groups | Number | gi Number |
| 4832 | 26S rRNA | Mucorales | M26190 | 168378 |
| 4833 | 18S rRNA | Mucorales | AF113430 | 6561325 |
| 4834 | 18S rRNA | Mucorales | AF113430 | 6561325 |
| 4835 | 18S rRNA | Mucorales | AF113430 | 6561325 |
| 4836 | 18S rRNA | Mucorales + Candida, | AF113430 | 6561325 |
| | | Aspergillus, Ajellomyces and | | |
| | | Fusarium | | |
| 4837 | b-tubulin | Scedosporium/Fusarium/ | AM261879 | 146328331 |
| | exon 5 | Aspergillus | | |

### EXAMPLE 2

### PCR Electrospray Ionization Mass Spectrometry (PCR/ESI-MS) for Identification of Clinically-Relevant Fungi and Yeast

Diagnosis of candidemia typically relies upon blood cultures, which often require several days for correct species identification. Diagnosis of invasive fungal infection (IFI) relies upon a consensus of clinical and laboratory criteria with certainty ranging from definite to probable or possible. Because definite diagnosis requires observation of the organism in tissue, a significant proportion of patients with IFI fall into the probable or possible categories.

The present example describes a single molecular assay based upon broad PCR amplification followed by mass spectrometry for the identification of a wide variety of fungi and yeast (Table 3).

TABLE 3

| | | | Genbank | Genbank |
|---|---|---|---|---|
| | | | reference | reference |
| Primer | | Molecular | sequence | Sequence |
| pair | Fungal target (s) | target | accession | gi |
| 3030 | Broad/Most fungi | LSU rRNA | X70659.1 | 671812 |
| 3031 | Broad/Most fungi | LSU rRNA | X70659.1 | 671812 |
| 3766 | Broad/Most fungi | LSU rRNA | X70659.1 | 671812 |
| 3816 | Broad/All fungi | 5.8S rRNA | AY342214.1 | 33469617 |
| 3865 | Candida albicans/ | mtDNA SSU | AF285261.1 | 12539616 |
| | tropicalis/ | rRNA | | |
| | orthopsilosis/ | | | |
| | metopsilosis | | | |
| 3867 | Candida glabrata | mtDNA SSU | NC_004691.1 | 29570601 |
| | | rRNA | | |
| 3875 | Candida spp. | GSC1 | D88815.1 | 2274846 |
| 3862 | Aspergillus spp. | mtDNA SSU | NC_007445.1 | 77019995 |
| | | rRNA | | |
| 4145 | Crytococcus | mtDNA SSU | NC_004336.1 | 24080109 |
| | neoformans/ | rRNA | | |
| | bacillisporus | | | |
| 4151 | Crytococcus | mtDNA cytB | AB105930.1 | 39725512 |
| | neoformans/ | | | |
| | bacillisporus | | | |
| 4158 | Coccidiodes | mtDNA SSU | AASO01004342.1 | 115299853 |
| | immitis/posadasii | rRNA | | |
| 4314 | Mucor/Rhizopus | mtDNA cytB | AB025730.1 | 32170402 |
| 4318 | Mucoraceae | SSU rRNA | AF113430.1 | 6561325 |
| | | (chromosomal) | | |
| 4325 | Ajellomyces | CHS2 (chitin | M82943.1 | 166959 |
| | capsulatus and | synthase) | | |
| | dermatitidis | | | |
| 4328 | Pneumocystis | mtDNA LSU | EF439817.1 | 126742335 |
| | jirovecii | rRNA | | |
| 4333 | Fusarium (solani | TOPOII | EU214571.1 | 162532533 |
| | and oxysporum) | (topoisomerase | | |
| | | II) | | |

Four broad-range PCR primers were selected within the 5.8S and 26S rRNAs for detection of fungi such that the product base compositions could be used to directly identify species or narrow possibilities to a range of species. An additional twelve narrowly-focused primer pairs were designed to identify specific groups by virtue of product amplification and identify species by virtue of product base compositions. Automated PCR/ESI-MS and data analysis provide base composition signatures that identify clinically-relevant species of fungi and yeast. The primers described in Table 1 above could be used in the methods described herein.

The assay detected a range of fungi and yeast that using 16 primer pairs, allowing 6 samples to be run on a 96-well plate. The assay broadly detects fungi and yeast, and identifies members of Candida spp. (speciating C. albicans, C. tropicalis, C. glabrata, C. parapsilosis, and C. krusei), Aspergillus spp., Cryptococcus neoformans, Mucorales, Coccidiodes immitis/posadasii, Ajellomyces spp. and Fusarium spp. A collection of clinically-relevant isolates have been tested and correctly identified with automated data analysis.

The molecular target of primer pair 3030 is a region of the LSU (25/26S rRNA) spanning L13-L20. The primers are designed to avoid amplifying human DNA, and to amplify a large proportion of Ascomycota and Basidiomycota. Genera that perform with primer pair 3030 include, but are not limited to: Candida, Aspergillus, Pichia, Penicillium, Cryptococcus, Saccharomyces, Armillaria, Porpidia, Mycosphaerella, Peltigera, Phyllactinia, Cladosporium, Debaryomyces, Peziza, Aschersonia, Ramaria, Hanseniaspora, Monascus, Kluyveromyces, Erysiphe, Kazachstania, Ajellomyces, Coccidioides, Hysterangium, Dekkera, Polyporus, Alternaria, Hyphodontia, Pestalotiopsis, Phaeosphaeria, Xanthoparmelia, Serpula, Emericella, Williopsis, Blumeria, Golovinomyces, Paracoccidioides, Zygosaccharomyces, Aureobasidium, Ophiostoma, Trametes, Arthroderma, Trichosporon, Antrodia, Botryosphaeria, Preussia, Thysanophora, Emmonsia, Fulgensia, Monacrosporium, Pseudocyphellaria, Harknessia, Neosartorya, Oidium, Eupenicillium and Phlebia. Members of >1100 more genera of Ascomycota and Basidiomycota are also in the set of organisms for amplification with primer pair 3030.

The molecular target of primer pair 3031 is a region of the LSU (25/26S rRNA) spanning L28-L31. The primers are designed to avoid amplifying human DNA, and to amplify a large proportion of Ascomycota and Basidiomycota. Genera that perform with primer pair 3131 include, but are not limited to: Peltigera, Phyllactinia, Phaeosphaeria, Mycosphaerella, Candida, Porpidia, Pertusaria, Peziza, Xanthoparmelia, Pestalotiopsis, Coccidioides, Fulgensia, Monacrosporium, Saccharomyces, Botryosphaeria, Aspergillus, Umbilicaria, Blumeria, Capronia, Golovinomyces, Arthrobotrys, Erysiphe, Usnea, Tilletia, Parmelina, Schizosaccharomyces, Oidium, Melanohalea, Pseudocyphellaria, Lecanora, Pseudocercospora, Teratosphaeria, Melanelixia, Ochrolechia, Pichia, Ramichloridium, Exophiala, Parmotrema, Cladosporium, Kluyveromyces, Lobaria, Acarospora, Mitrula, Orbilia, Trichophaea, Xylaria, Calicium, Dactylella, Hypotrachyna, Myxozyma, Pleospora, Arthroderma, Blastobotrys, Cryptococcus, Lipomyces, Moschella, Penicillium, Pyrenula, Sawadaea, Scutellinia, Apiospora, Caloplaca, Diplodia, Geopora, Paracoccidioides, Rhinocladiella, Uncinocarpus and Ajellomyces. Members of >700 more genera of Ascomycota and Basidiomycota are in the set of organisms that amplify with primer pair 3031.

The molecular target of primer pair 3766 is a region of the LSU (25/26S rRNA) spanning L78-L79. The primers are designed to avoid amplifying human DNA and to amplify a large proportion of Ascomycota and Basidiomycota. Genera that perform with primer pair 3766 include, but are not limited to: Phaeosphaeria, Candida, Coccidioides, Saccharomyces, Aspergillus, Metarhizium, Kluyveromyces, Fusarium oxysporum, Lipomyces, Myxozyma, Blastobotrys, Cryptococcus, Uncinocarpus, Eremothecium, Gibberella, Hypocrea, Cordyceps, Haematonectria, Lodderomyces, Wickerhamiella, Ajellomyces, Dekkera, Fomes, Ganoderma, Magnaporthe, Trametes, Zygozyma, Antrodia, Gaeumannomyces, Neosartorya, Penicillium, Pichia, Schizosaccharomyces, Sporopachydermia, Arxula, Brettanomyces, Buellia, Chaetomium, Coniophora, Kazachstania, Neurospora, Physconia, Poria, Sugiyamaella, Symbiotaphrina, Trigonopsis and Ustilago. Members of an additional 30 genera of Ascomycota and Basidiomycota are in the set of organisms that amplify with primer pair 3766.

The molecular target of primer pair 3816 is the highly-conserved 5.8S rRNA. The primers are designed to avoid amplifying human DNA. Primer pair 3816 amplifies nearly all fungal and yeast targets from all fungal phyla.

The molecular target of primer pair 3865 is the mitochondrial SSU rRNA gene. Primer pair 3865 is targeted to the Candida albicans group of Candida spp., including Candida albicans, C. tropicalis, C. parapsilosis, C. metapsilosis and C. orthopsilosis. Primer pair 3865 provides a base composition that, taken together with primer pairs 3030, 3031, 3766, 3816, 3867 and 3875 provides identification of Candida albicans group members to the species level.

The molecular target of primer pair 3867 is the mitochondrial SSU rRNA gene. Primer pair 3867 is targeted to Candida glabrata and other yeasts more closely related to C. glabrata than to C. albicans (e.g., C. castellii, C. kefyr, C. krusei, C. humilis, Saccharomyces spp., Kluveromyces spp., Pichia spp.). Primer pair 3876 provides a base composition that, taken together with primer pairs 3030, 3031, 3766, 3816, 3865 and 3875 provides identification of Candida glabrata and related yeasts to the species level.

The molecular target of primer pair 3875 is GSC1 (also referred to as FSK1, one of the catalytic subunits of β-1,3-glucan synthase). Primer pair 3875 amplifies the majority of Candida spp., as well as a variety of related yeasts, including Saccharomyces spp., Pichia spp., Kluyveromyces spp., and Debaryomyces spp. Primer pair 3875 amplifies, and provides a discriminating base composition for Cryptococcus neoformans species, including C. neoformans var. neoformans and C. bacillisporus (C. neoformans var. bacillispora). Primer pair 3875 discriminates between species, and taken together with primer pairs 3030, 3031, 3766, 3816, 3865 and 3867 provides identification of a variety of yeasts to the species level.

The molecular target of primer pair 3862 is the mitochondrial SSU rRNA gene. Primer pair 3862 is specific for the genus Aspergillus and Aspergillus spp. Primer pair 3862 provides a base composition that, taken together with primer pairs 3030, 3031, 3766 and 3816, provides identification of many members of Aspergillus spp. including for example, A. fumigatus to species level.

The molecular target of primer pair 4145 is the mitochondrial SSU rRNA gene. Primer pair 4145 is specific to Cryptococcus neoformans, and provides a base composition that differentiates C. neoformans from C. bacillisporus.

The molecular target of primer pair 4151 is the mitochondrial cytB (cytochrome B) gene. Primer pair 4151 is specific to Cryptococcus neoformans and provides a base composition that differentiates C. neoformans from C. bacillisporus.

The molecular target of primer pair 4158 is the mitochondrial SSU rRNA gene. Primer pair 4158 is specific to Coccidiodes and provides a base composition that differentiates C. immitis from C. posadasii.

The molecular target of primer pair 4314 is the mitochondrial cytB (cytochrome B) gene. Primer pair 4314 targets members of Mucor spp. and Rhizopus spp. and related Zygomycota (e.g. Cunninghamella, Zygorhynchus, Mycocladus). Together with primer pairs 3816 and 4318, a base composition is provided, for example, that places an organism into the order Mucorales, and to the genus level, for example, Mucor or Rhizopus.

The molecular target of primer pair 4318 is the chromosomal SSU rRNA, surrounding L44. Primer pair 4318 targets members of order Mucorales. Together with primer pairs 3816 and 4318, a base composition is provided that places an organism into the order Mucorales, and to the genus level, for example, Mucor or Rhizopus.

The molecular target of primer pair 4325 is the chitin synthase gene CHS2 of Ajellomyces spp. and provides a base composition that differentiates Ajellomyces capsulatus (Histoplasm capsulatum) from Ajellomyces dermatitidis (Blastomyces dermatitidis).

The molecular target of primer pair 4328 is the mitochondrial LSU rRNA gene. Primer pair 4328 is specific to Pneumocystis jirovecii (i.e., the human strain of Pneumocystis carinii) and other animal strains of P. carinii, with P. jirovecii producing a base composition distinct from other animal strains.

The molecular target of primer pair 4333 is the topoisomerase II gene (TOPOII). Primer pair 4333 is targeted to Fusarium oxysporum and Fusarium solani, and provides a base composition that differentiates between the two. Primer pair 4333 also amplifies Gibberella spp., with base compositions distinct from Fusarium spp.

### EXAMPLE 4

### De Novo Determination of Base Composition of Amplicons Using Molecular Mass Modified Deoxynucleotide Triphosphates

Because the molecular masses of the four natural nucleobases fall within a narrow molecular mass range (A=313.058, G=329.052, C=289.046, T=304.046, values in Daltons—See, Table 4), a source of ambiguity in assignment of base composition may occur as follows: two nucleic acid strands having different base composition may have a difference of about 1 Da when the base composition difference between the two strands is G⇄A (−15.994) combined with C⇄T (+15.000). For example, one 99-mer nucleic acid strand having a base composition of A₂₇G₃₀C₂₁T₂₁ has a theoretical molecular mass of 30779.058 while another 99-mer nucleic acid strand having a base composition of A₂₆G₃₁C₂₂T₂₀ has a theoretical molecular mass of 30780.052 is a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G⇄A combined with C⇄T event (Table 4). Thus, the same G⇄A (−15.994) event combined with 5-Iodo-C⇄T (−110.900) event would result in a molecular mass difference of 126.894 Da. The molecular mass of the base composition A₂₇G₃₀5-Iodo-C₂₁T₂₁ (33422.958) compared with A₂₆G₃₁5-Iodo-C₂₂T₂₀, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A₂₇G₃₀5-Iodo-C₂₁T₂₁. In contrast, the analogous amplification without the mass tag has 18 possible base compositions.

TABLE 4
Molecular Masses of Natural Nucleobases
and the Mass-Modified Nucleobase
5-Iodo-C and Molecular Mass
Differences Resulting from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons may be analyzed using a maximum-likelihood processor, as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-detection plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bioagents (e.g., species of fungi) and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring may be carried out as follows. Electronic PCR can be conducted on nucleotide sequences of the desired bioagents to obtain the different expected base counts that could be obtained for each primer pair. See for example, Schuler, Genome Res. 7:541-50, 1997; or the e-PCR program available from National Center for Biotechnology Information (NCBI, NIH, Bethesda, Md.). In one embodiment one or more spreadsheets from a workbook comprising a plurality of spreadsheets may be used (e.g., Microsoft Excel). First, in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by selecting the most abundant strain's base type composition and adding it to the reference set, and then the next most abundant strain's base type composition is added until the threshold is met or exceeded.

For each base count not included in the reference base count set for the bioagent of interest, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260 (U.S. application Ser. No. 10/418,514) which is incorporated herein by reference in entirety.

### EXAMPLE 5

### Primer Pair Panels for Identification of Fungi

This example illustrates an exemplary primer pair panel for identification of important groups of fungi. A first panel comprises 15 primer pairs designed to identify fungal groups including: Ascomycota, Basidiomycota, Candida, Aspergillus, Cryptococcus, Coccidioides, Mucorales, Ajellomyces and Pneumocystis. An exemplary assay format is adapted for a 96-well microtiter plate containing six duplicate PCR reactions for each of the 16 primer pairs. The primer pairs of this primer pair panel were previously described in Tables 1A to 1C and are shown in Table 5. Advantageously, this panel may include a control primer pair to ensure that the reactions operate as intended.

TABLE 5
Primer Pair Panel for Identification of Fungi

| Primer | |
|---|---|
| Pair | |
| Number | Primer Pair Name/Amplicon Coordinates |
| 3030 | 25SFUNG_X70659_134_261 |
| 3031 | 25SFUNG_X70659_697_834 |
| 3766 | 25SFUNG_X70659_2470_2617 |
| 3816 | 5P8SRNA_AY342214-165-322_34_111 |
| 3862 | ASP-MITSSURRNA_NC007445-8943-10314_13_120 |
| 3865 | CAN-MITSSURRNA_AF285261-27022-28483_844_958 |
| 3867 | CANGL-MITSSURRNA_NC004691-18404-20053_836_954 |
| 3875 | GSC1_D88815-723-6308_3141_3261 |
| 4145 | CRYPTO-MITSSURNA_NC004336-10422-11799_234_320 |
| 4151 | CRYPTO-MITCYTB_AB105930_1256_1356 |
| 4158 | COCCID-MIT_AASO01004342-13441-12000_326_419 |
| 4314 | MUCOR-MIT-CYTB_AB025730_92_216 |
| 4318 | MUCOR-18SRNA_AF113430_1646_1747 |
| 4325 | AJEL-CHS2_M82943_269_349 |
| 4328 | PNEUMO-MITLSURNA_EF439817_32_107 |

Shown in Table 6 is an alternate panel which was developed to provide increased coverage of molds. Primer pair numbers 3875, 4151, 4314, 4318 and 4325 were replaced by primer pair numbers 4837, 4832, 4835 and 4836. Groups included in this replacement coverage include Scedosporium and Mucorales at the expense of a confirmation primer pair for Ajellomyces, a primer pair for identification of yeast and a confirmation primer pair for Cryptococcus neoformans.

TABLE 6
Primer Pair Panel for Identification of Fungi

| Primer | |
|---|---|
| Pair | |
| Number | Primer Pair Name/Amplicon Coordinates |
| 3030 | 25SFUNG_X70659_134_261 |
| 3031 | 25SFUNG_X70659_697_834 |
| 3766 | 25SFUNG_X70659_2470_2617 |
| 3816 | 5P8SRNA_AY342214-165-322_34_111 |
| 3865 | CAN-MITSSURRNA_AF285261-27022-28483_844_958 |
| 3867 | CANGL-MITSSURRNA_NC004691-18404-20053_836_954 |
| 3862 | ASP-MITSSURRNA_NC007445-8943-10314_13_120 |
| 4145 | CRYPTO-MITSSURNA_NC004336-10422-11799_234_320 |
| 4158 | COCCID-MIT_AASO01004342-13441-12000_326_419 |
| 4837 | SCEDBTUB_AM261879_78_186 |
| 4318 | MUCOR-18SRNA_AF113430_1646_1747 |
| 4832 | 26SMUCOR_M26190_411_514 |
| 4835 | 18SMUCOR_AF113430_712_803 |
| 4836 | 18SMUCOR_AF113430_886_1021 |
| 4328 | PNEUMO-MITLSURNA_EF439817_32_107 |

Yet another primer pair panel is shown in Table 7 for increased coverage of molds at the expense of coverage of Coccidioides, Pneumocystis and Ajellomyces.

TABLE 7
Primer Pair Panel for Identification of Fungi

| Primer | |
|---|---|
| Pair | |
| Number | Primer Pair Name / Amplicon Coordinates |
| 3030 | 25SFUNG_X70659_134_261 |
| 3031 | 25SFUNG_X70659_697_834 |
| 3766 | 25SFUNG_X70659_2470_2617 |
| 3816 | 5P8SRNA_AY342214-165-322_34_111 |
| 3865 | CAN-MITSSURRNA_AF285261-27022-28483_844_958 |
| 3867 | CANGL-MITSSURRNA_NC004691-18404-20053_836_954 |
| 3875 | GSC1_D88815-723-6308_3141_3261 |
| 3862 | ASP-MITSSURRNA_NC007445-8943-10314_13_120 |
| 4145 | CRYPTO-MITSSURNA_NC004336-10422-11799_234_320 |
| 4151 | CRYPTO-MITCYTB_AB105930_1256_1356 |
| 4837 | SCEDBTUB_AM261879_78_186 |
| 4832 | 26SMUCOR_M26190_411_514 |
| 4318 | MUCOR-18SRNA_AF113430_1646_1747 |
| 4835 | 18SMUCOR_AF113430_712_803 |
| 4836 | 18SMUCOR_AF113430_886_1021 |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

We claim:

1. A composition, comprising at least one purified oligonucleotide primer pair that comprises forward and reverse primers, wherein said primer pair comprises nucleic acid sequences that are substantially complementary to nucleic acid sequences of two or more different bioagents belonging to the fungi kingdom, wherein said primer pair is configured to produce amplicons comprising different base compositions that correspond to said two or more different bioagents.

2. The composition of claim 1, wherein said primer pair is configured to hybridize with conserved regions of said two or more different bioagents and flank variable regions of said two or more different bioagents.

3. The composition of claim 1, wherein said forward and reverse primers are about 15 to 35 nucleobases in length, and wherein the forward primer comprises at least 70%, sequence identity with a sequence selected from the group consisting of SEQ ID NOs: 1-70, and the reverse primer comprises at least 70% sequence identity with a sequence selected from the group consisting of SEQ ID NOs: 71-140.

4. The composition of claim 1, wherein said primer pair is selected from the group of primer pair sequences consisting of: SEQ ID NOs: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, 70:140, 141:142, 143:144, 145:146, 147:148, 149:150, and 151:152.

5. The composition of claim 1, wherein said forward and reverse primers are about 15 to 35 nucleobases in length, and wherein:
the forward primer comprises at least 70% sequence identity with the sequence of SEQ ID NO: 1, and the reverse primer comprises at least 70% sequence identity with the sequence of SEQ ID NO: 71;
the forward primer comprises at least 70% sequence identity with the sequence of SEQ ID NO: 2, and the reverse primer comprises at least 70% sequence identity with the sequence of SEQ ID NO: 72;
the forward primer comprises at least 70% sequence identity with the sequence of SEQ ID NO: 3, and the reverse primer comprises at least 70% sequence identity with the sequence of SEQ ID NO: 73;
the forward primer comprises at least 70% sequence identity with the sequence of SEQ ID NO: 4, and the reverse primer comprises at least 70% sequence identity with the sequence of SEQ ID NO: 74;
the forward primer comprises at least 70% sequence identity with the sequence of SEQ ID NO: 5, and the reverse primer comprises at least 70% sequence identity with the sequence of SEQ ID NO: 75;
the forward primer comprises at least 70% sequence identity with the sequence of SEQ ID NO: 6, and the reverse primer comprises at least 70% sequence identity with the sequence of SEQ ID NO: 76;
the forward primer comprises at least 70% sequence identity with the sequence of SEQ ID NO: 7, and the reverse primer comprises at least 70% sequence identity with the sequence of SEQ ID NO: 77;
the forward primer comprises at least 70% sequence identity with the sequence of SEQ ID NO: 8, and the reverse primer comprises at least 70% sequence identity with the sequence of SEQ ID NO: 78;
the forward primer comprises at least 70% sequence identity with the sequence of SEQ ID NO: 9, and the reverse primer comprises at least 70% sequence identity with the sequence of SEQ ID NO: 79;
the forward primer comprises at least 70% sequence identity with the sequence of SEQ ID NO: 10, and the reverse primer comprises at least 70% sequence identity with the sequence of SEQ ID NO: 80;
the forward primer comprises at least 70% sequence identity with the sequence of SEQ ID NO: 11, and the reverse primer comprises at least 70% sequence identity with the sequence of SEQ ID NO: 81;
the forward primer comprises at least 70% sequence identity with the sequence of SEQ ID NO: 12, and the reverse primer comprises at least 70% sequence identity with the sequence of SEQ ID NO: 82;
the forward primer comprises at least 70% sequence identity with the sequence of SEQ ID NO: 13, and the reverse primer comprises at least 70% sequence identity with the sequence of SEQ ID NO: 83;
the forward primer comprises at least 70% sequence identity with the sequence of SEQ ID NO: 14, and the reverse primer comprises at least 70% sequence identity with the sequence of SEQ ID NO: 84;
the forward primer comprises at least 70% sequence identity with the sequence of SEQ ID NO: 15, and the reverse primer comprises at least 70% sequence identity with the sequence of SEQ ID NO: 85;
the forward primer comprises at least 70% sequence identity with the sequence of SEQ ID NO: 16, and the reverse primer comprises at least 70% sequence identity with the sequence of SEQ ID NO: 86;
the forward primer comprises at least 70% sequence identity with the sequence of SEQ ID NO: 17, and the reverse primer comprises at least 70% sequence identity with the sequence of SEQ ID NO: 87;
the forward primer comprises at least 70% sequence identity with the sequence of SEQ ID NO: 18, and the reverse primer comprises at least 70% sequence identity with the sequence of SEQ ID NO: 88;
the forward primer comprises at least 70% sequence identity with the sequence of SEQ ID NO: 19, and the reverse primer comprises at least 70% sequence identity with the sequence of SEQ ID NO: 89;
the forward primer comprises at least 70% sequence identity with the sequence of SEQ ID NO: 20, and the reverse primer comprises at least 70% sequence identity with the sequence of SEQ ID NO: 90;
the forward primer comprises at least 70% sequence identity with the sequence of SEQ ID NO: 21, and the reverse primer comprises at least 70% sequence identity with the sequence of SEQ ID NO: 91;
the forward primer comprises at least 70% sequence identity with the sequence of SEQ ID NO: 22, and the reverse primer comprises at least 70% sequence identity with the sequence of SEQ ID NO: 92;
the forward primer comprises at least 70% sequence identity with the sequence of SEQ ID NO: 23, and the reverse primer comprises at least 70% sequence identity with the sequence of SEQ ID NO: 93;
the forward primer comprises at least 70% sequence identity with the sequence of SEQ ID NO: 24, and the reverse primer comprises at least 70% sequence identity with the sequence of SEQ ID NO: 94;
the forward primer comprises at least 70% sequence identity with the sequence of SEQ ID NO: 25, and the reverse primer comprises at least 70% sequence identity with the sequence of SEQ ID NO: 95;
the forward primer comprises at least 70% sequence identity with the sequence of SEQ ID NO: 26, and the reverse primer comprises at least 70% sequence identity with the sequence of SEQ ID NO: 96;
the forward primer comprises at least 70% sequence identity with the sequence of SEQ ID NO: 27, and the reverse primer comprises at least 70% sequence identity with the sequence of SEQ ID NO: 97;
the forward primer comprises at least 70% sequence identity with the sequence of SEQ ID NO: 28, and the reverse primer comprises at least 70% sequence identity with the sequence of SEQ ID NO: 98;
the forward primer comprises at least 70% sequence identity with the sequence of SEQ ID NO: 29, and the reverse primer comprises at least 70% sequence identity with the sequence of SEQ ID NO: 99;
the forward primer comprises at least 70% sequence identity with the sequence of SEQ ID NO: 30, and the reverse primer comprises at least 70% sequence identity with the sequence of SEQ ID NO: 100;
the forward primer comprises at least 70% sequence identity with the sequence of SEQ ID NO: 31, and the reverse primer comprises at least 70% sequence identity with the sequence of SEQ ID NO: 101;
the forward primer comprises at least 70% sequence identity with the sequence of SEQ ID NO: 32, and the reverse primer comprises at least 70% sequence identity with the sequence of SEQ ID NO: 102;
the forward primer comprises at least 70% sequence identity with the sequence of SEQ ID NO: 33, and the reverse primer comprises at least 70% sequence identity with the sequence of SEQ ID NO: 103;
the forward primer comprises at least 70% sequence identity with the sequence of SEQ ID NO: 34, and the reverse primer comprises at least 70% sequence identity with the sequence of SEQ ID NO: 104;
the forward primer comprises at least 70% sequence identity with the sequence of SEQ ID NO: 35, and the reverse primer comprises at least 70% sequence identity with the sequence of SEQ ID NO: 105;
the forward primer comprises at least 70% sequence identity with the sequence of SEQ ID NO: 36, and the reverse primer comprises at least 70% sequence identity with the sequence of SEQ ID NO: 106;
the forward primer comprises at least 70% sequence identity with the sequence of SEQ ID NO: 37, and the reverse primer comprises at least 70% sequence identity with the sequence of SEQ ID NO: 107;
the forward primer comprises at least 70% sequence identity with the sequence of SEQ ID NO: 38, and the reverse primer comprises at least 70% sequence identity with the sequence of SEQ ID NO: 108;
the forward primer comprises at least 70% sequence identity with the sequence of SEQ ID NO: 39, and the reverse primer comprises at least 70% sequence identity with the sequence of SEQ ID NO: 109;
the forward primer comprises at least 70% sequence identity with the sequence of SEQ ID NO: 40, and the reverse primer comprises at least 70% sequence identity with the sequence of SEQ ID NO: 110;
the forward primer comprises at least 70% sequence identity with the sequence of SEQ ID NO: 41, and the reverse primer comprises at least 70% sequence identity with the sequence of SEQ ID NO: 111;
the forward primer comprises at least 70% sequence identity with the sequence of SEQ ID NO: 42, and the reverse primer comprises at least 70% sequence identity with the sequence of SEQ ID NO: 112;
the forward primer comprises at least 70% sequence identity with the sequence of SEQ ID NO: 43, and the reverse primer comprises at least 70% sequence identity with the sequence of SEQ ID NO: 113;
the forward primer comprises at least 70% sequence identity with the sequence of SEQ ID NO: 44, and the reverse primer comprises at least 70% sequence identity with the sequence of SEQ ID NO: 114;
the forward primer comprises at least 70% sequence identity with the sequence of SEQ ID NO: 45, and the reverse primer comprises at least 70% sequence identity with the sequence of SEQ ID NO: 115;
the forward primer comprises at least 70% sequence identity with the sequence of SEQ ID NO: 46, and the reverse primer comprises at least 70% sequence identity with the sequence of SEQ ID NO: 116;
the forward primer comprises at least 70% sequence identity with the sequence of SEQ ID NO: 47, and the reverse primer comprises at least 70% sequence identity with the sequence of SEQ ID NO: 117;
the forward primer comprises at least 70% sequence identity with the sequence of SEQ ID NO: 48, and the reverse primer comprises at least 70% sequence identity with the sequence of SEQ ID NO: 118;
the forward primer comprises at least 70% sequence identity with the sequence of SEQ ID NO: 49, and the reverse primer comprises at least 70% sequence identity with the sequence of SEQ ID NO: 119;
the forward primer comprises at least 70% sequence identity with the sequence of SEQ ID NO: 50, and the reverse primer comprises at least 70% sequence identity with the sequence of SEQ ID NO: 120;
the forward primer comprises at least 70% sequence identity with the sequence of SEQ ID NO: 51, and the reverse primer comprises at least 70% sequence identity with the sequence of SEQ ID NO: 121;
the forward primer comprises at least 70% sequence identity with the sequence of SEQ ID NO: 52, and the reverse primer comprises at least 70% sequence identity with the sequence of SEQ ID NO: 122;
the forward primer comprises at least 70% sequence identity with the sequence of SEQ ID NO: 53, and the reverse primer comprises at least 70% sequence identity with the sequence of SEQ ID NO: 123;
the forward primer comprises at least 70% sequence identity with the sequence of SEQ ID NO: 54, and the reverse primer comprises at least 70% sequence identity with the sequence of SEQ ID NO: 124;
the forward primer comprises at least 70% sequence identity with the sequence of SEQ ID NO: 55, and the reverse primer comprises at least 70% sequence identity with the sequence of SEQ ID NO: 125;
the forward primer comprises at least 70% sequence identity with the sequence of SEQ ID NO: 56, and the reverse primer comprises at least 70% sequence identity with the sequence of SEQ ID NO: 126;
the forward primer comprises at least 70% sequence identity with the sequence of SEQ ID NO: 57, and the reverse primer comprises at least 70% sequence identity with the sequence of SEQ ID NO: 127;
the forward primer comprises at least 70% sequence identity with the sequence of SEQ ID NO: 58, and the reverse primer comprises at least 70% sequence identity with the sequence of SEQ ID NO: 128;
the forward primer comprises at least 70% sequence identity with the sequence of SEQ ID NO: 59, and the reverse primer comprises at least 70% sequence identity with the sequence of SEQ ID NO: 129;
the forward primer comprises at least 70% sequence identity with the sequence of SEQ ID NO: 60, and the reverse primer comprises at least 70% sequence identity with the sequence of SEQ ID NO: 130;
the forward primer comprises at least 70% sequence identity with the sequence of SEQ ID NO: 61, and the reverse primer comprises at least 70% sequence identity with the sequence of SEQ ID NO: 131;
the forward primer comprises at least 70% sequence identity with the sequence of SEQ ID NO: 62, and the reverse primer comprises at least 70% sequence identity with the sequence of SEQ ID NO: 132;
the forward primer comprises at least 70% sequence identity with the sequence of SEQ ID NO: 63, and the reverse primer comprises at least 70% sequence identity with the sequence of SEQ ID NO: 133;
the forward primer comprises at least 70% sequence identity with the sequence of SEQ ID NO: 64, and the reverse primer comprises at least 70% sequence identity with the sequence of SEQ ID NO: 134;
the forward primer comprises at least 70% sequence identity with the sequence of SEQ ID NO: 65 and the reverse primer comprises at least 70% sequence identity with the sequence of SEQ ID NO: 135;
the forward primer comprises at least 70% sequence identity with the sequence of SEQ ID NO: 66, and the reverse primer comprises at least 70% sequence identity with the sequence of SEQ ID NO: 136;
the forward primer comprises at least 70% sequence identity with the sequence of SEQ ID NO: 67, and the reverse primer comprises at least 70% sequence identity with the sequence of SEQ ID NO: 137;
the forward primer comprises at least 70% sequence identity with the sequence of SEQ ID NO: 68, and the reverse primer comprises at least 70% sequence identity with the sequence of SEQ ID NO: 138;
the forward primer comprises at least 70% sequence identity with the sequence of SEQ ID NO: 69, and the reverse primer comprises at least 70% sequence identity with the sequence of SEQ ID NO: 139;
the forward primer comprises at least 70% sequence identity with the sequence of SEQ ID NO: 70, and the reverse primer comprises at least 70% sequence identity with the sequence of SEQ ID NO: 140;
the forward primer comprises at least 70% sequence identity with the sequence of SEQ ID NO: 141, and the reverse primer comprises at least 70% sequence identity with the sequence of SEQ ID NO: 142;
the forward primer comprises at least 70% sequence identity with the sequence of SEQ ID NO: 143, and the reverse primer comprises at least 70% sequence identity with the sequence of SEQ ID NO: 144;
the forward primer comprises at least 70% sequence identity with the sequence of SEQ ID NO: 145, and the reverse primer comprises at least 70% sequence identity with the sequence of SEQ ID NO: 146;
the forward primer comprises at least 70% sequence identity with the sequence of SEQ ID NO: 147, and the reverse primer comprises at least 70% sequence identity with the sequence of SEQ ID NO: 148;
the forward primer comprises at least 70% sequence identity with the sequence of SEQ ID NO: 149, and the reverse primer comprises at least 70% sequence identity with the sequence of SEQ ID NO: 150; and
the forward primer comprises at least 70% sequence identity with the sequence of SEQ ID NO: 151, and the reverse primer comprises at least 70% sequence identity with the sequence of SEQ ID NO: 152.

6. The composition of claim 1, wherein said different base compositions identify said two or more different bioagents at genus levels, species levels, sub-species levels or genotype levels.

7. The composition of claim 1, wherein said two or more amplicons are 45 to 200 nucleobases in length.

8. A kit comprising the composition of claim 1.

9. The composition of claim 1, wherein said different bioagents are selected from the group consisting of Candida spp., Aspergillus spp., Fusarium spp., Cryptococus spp., Coccidiodes spp., Mucor spp., Rhizopus spp., Mucorales, Ajellomyces spp., and Pneumocystis jirovecii.

10. The composition of claim 1, wherein a non-templated T residue on the 5′-end of said forward and/or reverse primer is removed.

11. The composition of claim 1, wherein said forward and/or reverse primer further comprises a non-templated T residue on the 5′-end.

12. The composition of claim 1, wherein said forward and/or reverse primer comprises at least one molecular mass modifying tag.

13. The composition of claim 1, wherein said forward and/or reverse primer comprises at least one modified nucleobase.

14. The composition of claim 13, wherein said modified nucleobase is 5-propynyluracil or 5-propynylcytosine.

15. The composition of claim 13, wherein said modified nucleobase is a mass modified nucleobase.

16. The composition of claim 15, wherein said mass modified nucleobase is 5-Iodo-C.

17. The composition of claim 13, wherein said modified nucleobase is a universal nucleobase.

18. The composition of claim 17, wherein said universal nucleobase is inosine.

19. A composition comprising an isolated oligonucleotide primer 15-35 bases in length selected form the group consisting of SEQ ID NOs 1-152.

20. A kit, comprising at least one purified oligonucleotide primer pair that comprises forward and reverse primers that are about 20 to 35 nucleobases in length, and wherein said forward primer comprises at least 70% sequence identity with a sequence selected from the group consisting of SEQ ID NOs: 1-70, 141, 143, 145, 147, 149, and 151 and said reverse primer comprises at least 70% sequence identity with a sequence selected from the group consisting of SEQ ID NOs: 71-140, 142, 144, 146, 148, 150 and 152.

21. The kit of claim 20, comprising at least one additional primer pair selected from the group of primer pair sequences consisting of: SEQ ID NOs: 2:72, 3:73, 5:75, 7:77, 14:84, 18:88, 20:90, 29:99, 37:107, 43:113, 44:114, 50:120, 54:120, 54:124, 61:131, 64:134, 69:139, 141:142, 143:144, 145:146, 147:148, 149:150 and 151:152.

22. A method of determining the presence of a fungus in at least one sample, the method comprising:
(a) amplifying one or more segments of at least one nucleic acid from said sample using at least one purified oligonucleotide primer pair that comprises forward and reverse primers that are about 20 to 35 nucleobases in length, and wherein said forward primer comprises at least 70% sequence identity with a sequence selected from the group consisting of SEQ ID NOs: 1-70, 141, 143, 145, 147, 149, and 151 and said reverse primer comprises at least 70% sequence identity with a sequence selected from the group consisting of SEQ ID NOs: 71-140, 142, 144, 146, 148, 150 and 152 to produce at least one amplification product; and
(b) detecting said amplification product, thereby determining said presence of said fungi in said sample.

23. The method of claim 22, wherein (a) comprises amplifying said one or more segments of said at least one nucleic acid from at least two samples obtained from different geographical locations to produce at least two amplification products, and (b) comprises detecting said amplification products, thereby tracking an epidemic spread of said fungi.

24. The method of claim 22, wherein (b) comprises determining an amount of said fungi in said sample.

25. The method of claim 22, wherein (b) comprises detecting a molecular mass of said amplification product.

26. The method of claim 22, wherein (b) comprises determining a base composition of said amplification product, wherein said base composition identifies the number of A residues, C residues, T residues, G residues, U residues, analogs thereof and/or mass tag residues thereof in said amplification product, whereby said base composition indicates the presence of fungi in said sample or identifies said fungi in said sample.

27. The method of claim 26, comprising comparing said base composition of said amplification product to calculated or measured base compositions of amplification products of one or more known fungi present in a database with the proviso that sequencing of said amplification product is not used to indicate the presence of or to identify said fungi, wherein a match between said determined base composition and said calculated or measured base composition in said database indicates the presence of or identifies said fungi.

28. A method of identifying one or more strains of fungi in a sample, the method comprising:
(a) amplifying two or more segments of a nucleic acid from said one or more fungi in said sample with first and second oligonucleotide primer pairs to obtain two or more amplification products, wherein said first primer pair is a broad range survey primer pair, and wherein said second primer pair is specific for an fungal gene selected from the group consisting of: 25S rRNA, actin, mito-ss rRNA, EFT-1a, EFT2, GSC1, pyroA, mito-ssu rRNA, 18S (ssu) rRNA, mito-cytochrome B, chitin synthase, mito-1su rRNA, major surface glycoprotein, beta-tubulin, and topoisomerase II;
(b) determining two or more molecular masses and/or base compositions of said two or more amplification products; and
(c) comparing said two or more molecular masses and/or said base compositions of said two or more amplification products with known molecular masses and/or known base compositions of amplification products of known fungi produced with said first and second primer pairs to identify said fungi in said sample.

29. The method of claim 28, wherein said second primer pair comprises forward and reverse primers that are about 20 to 35 nucleobases in length, and wherein said forward primer comprises at least 70% sequence identity with a sequence selected from the group consisting of SEQ ID NOs: 1-70, 141, 143, 145, 147, 149, and 151 and said reverse primer comprises at least 70% sequence identity with a sequence selected from the group consisting of SEQ ID NOs: 71-140, 142, 144, 146, 148, 150 and 152 to produce at least one amplification product.

30. The method of claim 29, wherein said first primer pair amplifies ribosomal RNA encoding sequences.

31. The method of claim 28, comprising obtaining said two or more molecular masses of said two or more amplification products via mass spectrometry.

31. The method of claim 28, comprising calculating said two or more base compositions from said two or more molecular masses of said two or more amplification products.

32. The method of claim 28, wherein said fungi is selected from the group consisting of: Candida spp., Aspergillus spp., Fusarium spp., Cryptococus spp., Coccidiodes spp., Mucor spp., Rhizopus spp., Mucorales, Ajellomyces spp., and Pneumocystis jirovecii.

33. The method of claim 28, wherein said two or more primer pairs comprise two or more purified oligonucleotide primer pairs that each comprise forward and reverse primers that are about 20 to 35 nucleobases in length, and wherein said forward primers comprise at least 70% sequence identity with a sequence selected from the group consisting of SEQ ID NOs: 1-70, 141, 143, 145, 147, 149, and 151 and said reverse primers comprise at least 70% sequence identity with a sequence selected from the group consisting of SEQ ID NOs: 71-140, 142, 144, 146, 148, 150 and 152 to obtain an amplification product.

35. The method of claim 28, wherein said primer pairs are selected from the group of primer pair sequences consisting of: SEQ ID NOs: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, 70:140, 141:142, 143:144, 145:146, 147:148, 149:150, and 151:152.

36. The method of claim 28, wherein said primer pairs consist of: SEQ ID NOs: 2:72, 3:73, 5:75, 7:77, 14:84, 18:88, 20:90, 29:99, 37:107, 43:113, 44:114, 50:120, 54:120, 54:124, 61:131, 64:134, 69:139, 141:142, 143:144, 145:146, 147:148, 149:150 and 151:152.

37. The method of claim 28, wherein said determining said two or more molecular masses and/or base compositions is conducted without sequencing said two or more amplification products.

38. The method of claim 28, wherein said one or more fungi bioagents in said sample cannot be identified using a single primer pair of said two or more primer pairs.

39. The method of claim 28, wherein said one or more fungi bioagents in a sample are identified by comparing three or more molecular masses and/or base compositions of three or more amplification products with a database of known molecular masses and/or known base compositions of amplification products of known fungi bioagents produced with said three or more primer pairs.

40. The method of claim 28, wherein said two or more segments of said nucleic acid are amplified from a single gene.

41. The method of claim 28, wherein said two or more segments of said nucleic acid are amplified from different genes.

42. The method of claim 28, wherein members of said primer pairs hybridize to conserved regions of said nucleic acid that flank a variable region.

43. The method of claim 42, wherein said variable region varies between at least two of said fungi bioagents.

44. The method of claim 42, wherein said variable region uniquely varies between at least five of said fungi bioagents.

45. The method of claim 28, wherein said two or more amplification products obtained in (a) comprise major classification and subgroup identifying amplification products.

46. The method of claim 45, comprising comparing said molecular masses and/or said base compositions of said two or more amplification products to calculated or measured molecular masses or base compositions of amplification products of known fungal bioagents in a database comprising genus specific amplification products, species specific amplification products, strain specific amplification products or nucleotide polymorphism specific amplification products produced with said two or more oligonucleotide primer pairs, wherein one or more matches between said two or more amplification products and one or more entries in said database identifies said one or more fungal bioagents, classifies a major classification of said one or more fungal bioagents, and/or differentiates between subgroups of known and unknown fungal bioagents in said sample.

47. The method of claim 46, wherein said major classification of said one or more fungal bioagents comprises a genus or species classification of said one or more fungal bioagents.

48. The method of claim 46, wherein said subgroups of known and unknown fungal bioagents comprise family, strain and nucleotide variations of said one or more fungal bioagents.

49. A system, comprising:
(a) a mass spectrometer configured to detect one or more molecular masses of amplicons produced using at least one purified oligonucleotide primer pair that comprises forward and reverse primers, wherein said primer pair comprises nucleic acid sequences that are substantially complementary to nucleic acid sequences of two or more different fungal bioagents; and
(b) a controller operably connected to said mass spectrometer, said controller configured to correlate said molecular masses of said amplicons with one or more fungi bioagent identities.

50. The system of claim 49, wherein said fungal bioagent identities are at genus, species, and/or sub-species levels.

51. The system of claim 49, wherein said forward and reverse primers are about 15 to 35 nucleobases in length, and wherein the forward primer comprises at least 70% sequence identity with a sequence selected from the group consisting of SEQ ID NOs: 1-70, 141, 143, 145, 147, 149 and 151 and the reverse primer comprises at least 70% sequence identity with a sequence selected from the group consisting of SEQ ID NOS: 70-140, 142, 144, 146, 148, 150 and 152.

52. The system of claim 49, wherein said primer pair is selected from the group of primer pair sequences consisting of: SEQ ID NOs: 1:71, 2:72, 3:73, 4:74, 5:75, 6:76, 7:77, 8:78, 9:79, 10:80, 11:81, 12:82, 13:83, 14:84, 15:85, 16:86, 17:87, 18:88, 19:89, 20:90, 21:91, 22:92, 23:93, 24:94, 25:95, 26:96, 27:97, 28:98, 29:99, 30:100, 31:101, 32:102, 33:103, 34:104, 35:105, 36:106, 37:107, 38:108, 39:109, 40:110, 41:111, 42:112, 43:113, 44:114, 45:115, 46:116, 47:117, 48:118, 49:119, 50:120, 51:121, 52:122, 53:123, 54:124, 55:125, 56:126, 57:127, 58:128, 59:129, 60:130, 61:131, 62:132, 63:133, 64:134, 65:135, 66:136, 67:137, 68:138, 69:139, 70:140, 141:142, 143:144, 145:146, 147:148, 149:150, and 151:152.

53. The system of claim 49, comprising at least one additional primer pair selected from the group of primer pair sequences consisting of: SEQ ID NOs: 2:72, 3:73, 5:75, 7:77, 14:84, 18:88, 20:90, 29:99, 37:107, 43:113, 44:114, 50:120, 54:120, 54:124, 61:131, 64:134, and 69:139, 141:142, 143:144, 145:146, 147:148, 149:150 and 151:152.

54. The system of claim 49, wherein said controller is configured to determine base compositions of said amplicons from said molecular masses of said amplicons, which base compositions correspond to said one or more fungal bioagent identities.

55. The system of claim 49, wherein said controller comprises or is operably connected to a database of known molecular masses and/or known base compositions of amplicons of known fungal bioagents produced with the primer pair.

56. A purified oligonucleotide primer pair, comprising a forward primer and a reverse primer that each independently comprise 14 to 40 consecutive nucleobases selected from the primer pair sequences shown in Table 1, which primer pair is configured to generate an amplicon between about 50 and 150 consecutive nucleobases in length.


SEQUENCE LISTING (152 sequences)

SEQ ID NO: 1 - DNA, 27 bp, Artificial Sequence
  Primer
tctcaggatagcagaagctcgtatcag

SEQ ID NO: 2 - DNA, 25 bp, Artificial Sequence
  Primer
tgtgaagcggcaaaagctcaaattt

SEQ ID NO: 3 - DNA, 26 bp, Artificial Sequence
  Primer
tggagtctaacatctatgcgagtgtt

SEQ ID NO: 4 - DNA, 25 bp, Artificial Sequence
  Primer
ttgtagaataggtgggagcttcggc

SEQ ID NO: 5 - DNA, 27 bp, Artificial Sequence
  Primer
ttgtgtagaataggtgggagcttcggc

SEQ ID NO: 6 - DNA, 24 bp, Artificial Sequence
  Primer
tcgatgaagaacgcagcgaaatgc

SEQ ID NO: 7 - DNA, 24 bp, Artificial Sequence
  Primer
tcgatgaagaacgcagcgaaatgc

SEQ ID NO: 8 - DNA, 30 bp, Artificial Sequence
  Primer
tggtccttgaaaatccacaggaaggaatag

SEQ ID NO: 9 - DNA, 26 bp, Artificial Sequence
  Primer
tcccgtacgcgtaatgaaagtgaacg

SEQ ID NO: 10 - DNA, 30 bp, Artificial Sequence
  Primer
tgtcaaacccgtacgcgtaatgaaagtgaa

SEQ ID NO: 11 - DNA, 27 bp, Artificial Sequence
  Primer
tgattcagagctccagaagctttgttc

SEQ ID NO: 12 - DNA, 23 bp, Artificial Sequence
  Primer
tcgatcgaccacattgggtctga

SEQ ID NO: 13 - DNA, 28 bp, Artificial Sequence
  Primer
tccttctatgtaaaaactgacgttgagg

SEQ ID NO: 14 - DNA, 26 bp, Artificial Sequence
  Primer
tccaagtacttgacacatgctaatcg

SEQ ID NO: 15 - DNA, 29 bp, Artificial Sequence
  Primer
tggattagataccctagtagtccaagcag

SEQ ID NO: 16 - DNA, 36 bp, Artificial Sequence
  Primer
tttcatatatttcgcactaatcactcatcaatagca

SEQ ID NO: 17 - DNA, 30 bp, Artificial Sequence
  Primer
tcggatgaaatgcaagacattagacggtta

SEQ ID NO: 18 - DNA, 30 bp, Artificial Sequence
  Primer
tggtacagtggagtatgctgtttaattgga

SEQ ID NO: 19 - DNA, 26 bp, Artificial Sequence
  Primer
tgcgacatggattcggtacccattta

SEQ ID NO: 20 - DNA, 30 bp, Artificial Sequence
  Primer
ttcgatacccgtgtagttctagtagtaaac

SEQ ID NO: 21 - DNA, 30 bp, Artificial Sequence; identical to SEQ ID NO: 20
  Primer

SEQ ID NO: 22 - DNA, 28 bp, Artificial Sequence
  Primer
tgtcccaggtgacaatgttggtttcaac

SEQ ID NO: 23 - DNA, 34 bp, Artificial Sequence
  Primer
tgccattgcaagatgtttacaagattggtggtat

SEQ ID NO: 24 - DNA, 31 bp, Artificial Sequence
  Primer
tagaagaggtaacgtttgtggtgactccaag

SEQ ID NO: 25 - DNA, 28 bp, Artificial Sequence
  Primer
tgtcccaggtgacaatgttggtttcaac

SEQ ID NO: 26 - DNA, 24 bp, Artificial Sequence
  Primer
tggtttacacggttgggctttcac

SEQ ID NO: 27 - DNA, 27 bp, Artificial Sequence
  Primer
tgtcgaaggtttgaagagattgtccaa

SEQ ID NO: 28 - DNA, 21 bp, Artificial Sequence
  Primer
tgtcattgcccacgtcgatca

SEQ ID NO: 29 - DNA, 27 bp, Artificial Sequence
  Primer
tgccttgattgacggtcattgtgaaat

SEQ ID NO: 30 - DNA, 24 bp, Artificial Sequence
  Primer
tgactacatcgacgagtccgaagt

SEQ ID NO: 31 - DNA, 26 bp, Artificial Sequence
  Primer
ttttttcccttcgtctgtggttgccg

SEQ ID NO: 32 - DNA, 24 bp, Artificial Sequence
  Primer
tcaaggatgctggcgtaatggtca

SEQ ID NO: 33 - DNA, 18 bp, Artificial Sequence
  Primer
ttccctggaacgggacgc

SEQ ID NO: 34 - DNA, 23 bp, Artificial Sequence
  Primer
tacaatgagctccgtgtcgctcc

SEQ ID NO: 35 - DNA, 31 bp, Artificial Sequence
  Primer
tcagtctaagcgaggtattcttaccttgaag

SEQ ID NO: 36 - DNA, 27 bp, Artificial Sequence
  Primer
tacagtcgaaagaccgaaacgtcaatg

SEQ ID NO: 37 - DNA, 23 bp, Artificial Sequence
  Primer
taccaagccaatgacgagtaggg

SEQ ID NO: 38 - DNA, 25 bp, Artificial Sequence
  Primer
tagtccagtctaatctccgtgccag

SEQ ID NO: 39 - DNA, 23 bp, Artificial Sequence
  Primer
tcgattcacttgcctctcagcag

SEQ ID NO: 40 - DNA, 18 bp, Artificial Sequence
  Primer
tcgaggcggtcctcctca

SEQ ID NO: 41 - DNA, 23 bp, Artificial Sequence
  Primer
tagtcgaacttcaggtctggcga

SEQ ID NO: 42 - DNA, 31 bp, Artificial Sequence
  Primer
tagcagctctagctctagtacatatgctaac

SEQ ID NO: 43 - DNA, 30 bp, Artificial Sequence
  Primer
taccatttattctagcagctctagctctag

SEQ ID NO: 44 - DNA, 21 bp, Artificial Sequence
  Primer
tcaatggcctaacggctgaac

SEQ ID NO: 45 - DNA, 19 bp, Artificial Sequence
  Primer
tggcctaacggctgaacca

SEQ ID NO: 46 - DNA, 28 bp, Artificial Sequence
  Primer
tgaaccagcaacttggaagaatgagaag

SEQ ID NO: 47 - DNA, 25 bp, Artificial Sequence
  Primer
tgaaccagcaacttggaagaatgag

SEQ ID NO: 48 - DNA, 32 bp, Artificial Sequence
  Primer
tggatctagtaatccactaggaattacagcaa

SEQ ID NO: 49 - DNA, 29 bp, Artificial Sequence
  Primer
tgcaacattaaaccgattcttcagtcttc

SEQ ID NO: 50 - DNA, 31 bp, Artificial Sequence
  Primer
ttcttcagtcttcactacttattaccattca

SEQ ID NO: 51 - DNA, 25 bp, Artificial Sequence
  Primer
tgttgcagttaaaacgtccgtagtc

SEQ ID NO: 52 - DNA, 23 bp, Artificial Sequence
  Primer
tgaggtggcctggtcttcattga

SEQ ID NO: 53 - DNA, 26 bp, Artificial Sequence
  Primer
tgcaggcttttaagcttgaatgtgtt

SEQ ID NO: 54 - DNA, 30 bp, Artificial Sequence
  Primer
tcgattgaatggttatagtgagcatatggg

SEQ ID NO: 55 - DNA, 24 bp, Artificial Sequence
  Primer
taatttagcggatcgcatggcctt

SEQ ID NO: 56 - DNA, 33 bp, Artificial Sequence
  Primer
tgacgttttcattgatcaaggtctaaagttaag

SEQ ID NO: 57 - DNA, 31 bp, Artificial Sequence
  Primer
tggttaattcctttaattaacgcaaaccctt

SEQ ID NO: 58 - DNA, 36 bp, Artificial Sequence
  Primer
tcatgctaatcgtacgacatcatattaaaaatgatg

SEQ ID NO: 59 - DNA, 34 bp, Artificial Sequence
  Primer
tgaaccagcaacttggaggaataaatgtaatgat

SEQ ID NO: 60 - DNA, 24 bp, Artificial Sequence
  Primer
tcatgggcgtctatcaggatggga

SEQ ID NO: 61 - DNA, 30 bp, Artificial Sequence
  Primer
tggatcttaaattcaaaggtgcagagaagg

SEQ ID NO: 62 - DNA, 24 bp, Artificial Sequence
  Primer
tgtctgaaggagcgcaatgagaag

SEQ ID NO: 63 - DNA, 23 bp, Artificial Sequence
  Primer
tcgatggttcttcaacgctttcg

SEQ ID NO: 64 - DNA, 28 bp, Artificial Sequence
  Primer
tttctgcgaaaattgttttggcaaattg

SEQ ID NO: 65 - DNA, 34 bp, Artificial Sequence
  Primer
tgcgaaaattgttttggcaaattgtttattcctc

SEQ ID NO: 66 - DNA, 26 bp, Artificial Sequence
  Primer
tgccagggatgccctgtttctttttg

SEQ ID NO: 67 - DNA, 21 bp, Artificial Sequence
  Primer
tcgcaggttggttgcgctttg

SEQ ID NO: 68 - DNA, 25 bp, Artificial Sequence
  Primer
tcgcatgagcgtctacttcaacgag

SEQ ID NO: 69 - DNA, 31 bp, Artificial Sequence
  Primer
tcaactgttacatcaacaatccagcttttac

SEQ ID NO: 70 - DNA, 22 bp, Artificial Sequence
  Primer
taagcgcaagggtcatgctctg

SEQ ID NO: 71 - DNA, 26 bp, Artificial Sequence
  Primer
tccacgttcaattaagcaacaaggac

SEQ ID NO: 72 - DNA, 27 bp, Artificial Sequence
  Primer
ttctcaccctctgtgacggcctgttcc

SEQ ID NO: 73 - DNA, 27 bp, Artificial Sequence
  Primer
tcagctatgctcttactcaaatccatc

SEQ ID NO: 74 - DNA, 23 bp, Artificial Sequence
  Primer
tgacaatgtcttcaacccggatc

SEQ ID NO: 75 - DNA, 25 bp, Artificial Sequence
  Primer
tctgacaatgtcttcaacccggatc

SEQ ID NO: 76 - DNA, 20 bp, Artificial Sequence
  Primer
tggcatgccctccggaatac

SEQ ID NO: 77 - DNA, 27 bp, Artificial Sequence
  Primer
tgtgcgttcaaagattcgatgattcac

SEQ ID NO: 78 - DNA, 35 bp, Artificial Sequence
  Primer
tgccgacttcccttatctacattattctatcaact

SEQ ID NO: 79 - DNA, 29 bp, Artificial Sequence
  Primer
tcccaacagctatgctcttactcaaatcc

SEQ ID NO: 80 - DNA, 29 bp, Artificial Sequence
  Primer
tcccaacagctacgctcttactcaaatcc

SEQ ID NO: 81 - DNA, 28 bp, Artificial Sequence
  Primer
tggagttgaaagtggtttggtcaatacc

SEQ ID NO: 82 - DNA, 26 bp, Artificial Sequence
  Primer
tcctttcggccattgaccaatattcc

SEQ ID NO: 83 - DNA, 30 bp, Artificial Sequence
  Primer
tgacattcatcattttctgcttggactact

SEQ ID NO: 84 - DNA, 29 bp, Artificial Sequence
  Primer
tccccttactttaaggtagccaaattatc

SEQ ID NO: 85 - DNA, 27 bp, Artificial Sequence
  Primer
tgccatattactcttgaggtggaatgc

SEQ ID NO: 86 - DNA, 32 bp, Artificial Sequence
  Primer
tgcccgaactgtatttcaacttatagcatatc

SEQ ID NO: 87 - DNA, 25 bp, Artificial Sequence
  Primer
tctgacgacaacaatgtaacgcctg

SEQ ID NO: 88 - DNA, 25 bp, Artificial Sequence
  Primer
tctgacgacaacaatgtaacgcctg

SEQ ID NO: 89 - DNA, 22 bp, Artificial Sequence
  Primer
tcggacgtactagtctggcggt

SEQ ID NO: 90 - DNA, 29 bp, Artificial Sequence
  Primer
tcattattgctaacgtactcttcaggtgg

SEQ ID NO: 91 - DNA, 31 bp, Artificial Sequence
  Primer
tgtctgtaaccgtctattgttttgagtttca

SEQ ID NO: 92 - DNA, 26 bp, Artificial Sequence
  Primer
tttggtggatcgttcttggagtcacc

SEQ ID NO: 93 - DNA, 29 bp, Artificial Sequence
  Primer
tcagctggggcgaaagtgacaaccatacc

SEQ ID NO: 94 - DNA, 30 bp, Artificial Sequence
  Primer
tgtgtgacaatccaagactggagagtaacc

SEQ ID NO: 95 - DNA, 26 bp, Artificial Sequence
  Primer
tttcgtggatcgttcttggagtcacc

SEQ ID NO: 96 - DNA, 27 bp, Artificial Sequence
  Primer
tgttagtccatttcttggtctttgggt

SEQ ID NO: 97 - DNA, 29 bp, Artificial Sequence
  Primer
tgcaaacaaatttccaagtgcaattcacc

SEQ ID NO: 98 - DNA, 34 bp, Artificial Sequence
  Primer
tacctctttcttgttcatcttttctggtatccat

SEQ ID NO: 99 - DNA, 33 bp, Artificial Sequence
  Primer
tgcatggttttggttatcagatttaccatcacc

SEQ ID NO: 100 - DNA, 21 bp, Artificial Sequence
  Primer
taggttgcggcagccacagac

SEQ ID NO: 101 - DNA, 18 bp, Artificial Sequence
  Primer
tggtgccggcctcaccct

SEQ ID NO: 102 - DNA, 20 bp, Artificial Sequence
  Primer
tcgaagctctcacctgcgtt

SEQ ID NO: 103 - DNA, 25 bp, Artificial Sequence
  Primer
tgcattcccaaactactcgactcgt

SEQ ID NO: 104 - DNA, 26 bp, Artificial Sequence
  Primer
tgtcatcttctctcggttctgcttgg

SEQ ID NO: 105 - DNA, 21 bp, Artificial Sequence
  Primer
tcaggagcgacacggagctca

SEQ ID NO: 106 - DNA, 23 bp, Artificial Sequence
  Primer
tacgcgggctatcggattaaacg

SEQ ID NO: 107 - DNA, 26 bp, Artificial Sequence
  Primer
tgctgcacataatctatgctctggac

SEQ ID NO: 108 - DNA, 28 bp, Artificial Sequence
  Primer
tacacctattcaaaactccgatgggttg

SEQ ID NO: 109 - DNA, 25 bp, Artificial Sequence
  Primer
tcactctccctactcgtcattggct

SEQ ID NO: 110 - DNA, 21 bp, Artificial Sequence
  Primer
tccaggaggtaaggtccagca

SEQ ID NO: 111 - DNA, 22 bp, Artificial Sequence
  Primer
tggaccaccaggaggtaaggtc

SEQ ID NO: 112 - DNA, 29 bp, Artificial Sequence
  Primer
taaagataaagtatggatgcattggtgcc

SEQ ID NO: 113 - DNA, 27 bp, Artificial Sequence
  Primer
tgccttttcagcatttgagctaatacc

SEQ ID NO: 114 - DNA, 32 bp, Artificial Sequence
  Primer
tccctagaatttcatacgatatattcgtttcg

SEQ ID NO: 115 - DNA, 25 bp, Artificial Sequence
  Primer
tgctggcacgtaatttggtcaagac

SEQ ID NO: 116 - DNA, 25 bp, Artificial Sequence
  Primer
tgctggcacgtaatttggtcaagac

SEQ ID NO: 117 - DNA, 32 bp, Artificial Sequence; identical to SEQ ID NO: 114
  Primer

SEQ ID NO: 118 - DNA, 36 bp, Artificial Sequence
  Primer
ttgggtgcatagaataagaataaagctaatactaag

SEQ ID NO: 119 - DNA, 31 bp, Artificial Sequence
  Primer
taaaccctatcagcatttgctgtaattccta

SEQ ID NO: 120 - DNA, 31 bp, Artificial Sequence; identical to SEQ ID NO: 119
  Primer

SEQ ID NO: 121 - DNA, 26 bp, Artificial Sequence
  Primer
tgcgagcttgatcaatgaagaccagg

SEQ ID NO: 122 - DNA, 32 bp, Artificial Sequence
  Primer
tgctttaaacactctgatttgctcatggtaat

SEQ ID NO: 123 - DNA, 28 bp, Artificial Sequence
  Primer
tttcacctctagcgaccaaatacaaatg

SEQ ID NO: 124 - DNA, 30 bp, Artificial Sequence
  Primer
tcttcctctaaatagcctagtttgccatag

SEQ ID NO: 125 - DNA, 27 bp, Artificial Sequence
  Primer
tcatgatagggcagaaaatcgagtgga

SEQ ID NO: 126 - DNA, 32 bp, Artificial Sequence
  Primer
tcaatctctagtcggcatagtttgtggttaag

SEQ ID NO: 127 - DNA, 29 bp, Artificial Sequence
  Primer
tccctattctctaaccatttttttagcga

SEQ ID NO: 128 - DNA, 28 bp, Artificial Sequence
  Primer
tggcatcttaaagcgaaaccttagtttc

SEQ ID NO: 129 - DNA, 25 bp, Artificial Sequence
  Primer
tgctggcacgtaatttggtcaagac

SEQ ID NO: 130 - DNA, 31 bp, Artificial Sequence
  Primer
tcccttctctgcacctttaaatttaagatcc

SEQ ID NO: 131 - DNA, 25 bp, Artificial Sequence
  Primer
tcttctcattgcgctccttcagaca

SEQ ID NO: 132 - DNA, 22 bp, Artificial Sequence
  Primer
tcgcccgaaagcgttgaagaac

SEQ ID NO: 133 - DNA, 24 bp, Artificial Sequence
  Primer
tgatcgaacgctttccagagatgg

SEQ ID NO: 134 - DNA, 26 bp, Artificial Sequence
  Primer
tccctcgagatattcagtgctatacc

SEQ ID NO: 135 - DNA, 33 bp, Artificial Sequence
  Primer
tccctgagataaatattttcatactccctcgag

SEQ ID NO: 136 - DNA, 25 bp, Artificial Sequence
  Primer
taccgaatacagggttcatgccgag

SEQ ID NO: 137 - DNA, 27 bp, Artificial Sequence
  Primer
tcatacctcgcacctcttcaaaatctg

SEQ ID NO: 138 - DNA, 22 bp, Artificial Sequence
  Primer
taccgggctcaagatcgacgag

SEQ ID NO: 139 - DNA, 26 bp, Artificial Sequence
  Primer
tcagtttgcatttgctaccaaactgg

SEQ ID NO: 140 - DNA, 29 bp, Artificial Sequence
  Primer
tctcctttgtttgtgaagtaaaagctgga

SEQ ID NO: 141 - DNA, 24 bp, Artificial Sequence
  Primer
tccgctttcaaagagtcaggttgt

SEQ ID NO: 142 - DNA, 26 bp, Artificial Sequence
  Primer
tggtacttgttcgctatcggtctctc

SEQ ID NO: 143 - DNA, 28 bp, Artificial Sequence
  Primer
tcttggattgaccgaagacaaactactg

SEQ ID NO: 144 - DNA, 26 bp, Artificial Sequence
  Primer
tcggtatctaatcgtcttcgatccct

SEQ ID NO: 145 - DNA, 31 bp, Artificial Sequence
  Primer
tcgaagacgattagataccgtcgtagtctta

SEQ ID NO: 146 - DNA, 31 bp, Artificial Sequence
  Primer
tccagaacccaaaaactttactttcgctaag

SEQ ID NO: 147 - DNA, 25 bp, Artificial Sequence
  Primer
tccagcggattaccatgagcaaatc

SEQ ID NO: 148 - DNA, 33 bp, Artificial Sequence
  Primer
tcctagagtcgtatttcattattccatgctaac

SEQ ID NO: 149 - DNA, 28 bp, Artificial Sequence
  Primer
tcttggattgaccgaagacaaactactg

SEQ ID NO: 150 - DNA, 28 bp, Artificial Sequence
  Primer
tctctagtcggcatcgtttgtggttaag

SEQ ID NO: 151 - DNA, 21 bp, Artificial Sequence
  Primer
tgccgcggtgctcactctttc

SEQ ID NO: 152 - DNA, 22 bp, Artificial Sequence
  Primer
tgtatcggccgttgcggaagtc